SEQUENCE LISTING (8 sequences)

SEQ ID NO: 1 - DNA, 20 bp, synthetic construct
  source 1..20: mol_type = other DNA
agaacatcatccctgcatcc

SEQ ID NO: 2 - DNA, 20 bp, synthetic construct
  source 1..20: mol_type = other DNA
ggtcctcagtgtagcccaag

SEQ ID NO: 3 - DNA, 20 bp, synthetic construct
  source 1..20: mol_type = other DNA
tcgtgctgtcggacccatat

SEQ ID NO: 4 - DNA, 22 bp, synthetic construct
  source 1..22: mol_type = other DNA
gtcgttgcttggttctccttgt

SEQ ID NO: 5 - DNA, 22 bp, synthetic construct
  source 1..22: mol_type = other DNA
acaaccacggccttccctactt

SEQ ID NO: 6 - DNA, 23 bp, synthetic construct
  source 1..23: mol_type = other DNA
cacgatttcccagagaacatgtg

SEQ ID NO: 7 - DNA, 17 bp, synthetic construct
  source 1..17: mol_type = other DNA
gccaccacgctcttctg

SEQ ID NO: 8 - DNA, 17 bp, synthetic construct
  source 1..17: mol_type = other DNA
ggtgtgggtgaggagca

(19) United States
(12) Patent Application Publication
(10) Pub. No.: US 2026/0092043 A1
(43) Pub. Date: Apr. 2, 2026

(54) NOVEL BENZOXAZOLE DERIVATIVE COMPRISING 4-AMINO-BUTANAMIDE AND USE THEREOF

(71) Applicant: EWHA UNIVERSITY - INDUSTRY COLLABORATION FOUNDATION, Seoul (KR)

(72) Inventors: Hyun Ae WOO, Seoul (KR); Hea Young PARK, Seoul (KR); Da-Rong KIM, Daegu (KR); Ji-Young PARK, Seoul (KR); Ji Hye YOO, Cheongju-si (KR); Yeon-ju HEO, Gwangmyeong-si (KR)

(73) Assignee: EWHA UNIVERSITY - INDUSTRY COLLABORATION FOUNDATION, (omitted)

(21) Appl. No.: 19/115,423

(22) PCT Filed: Mar. 14, 2023
(86) PCT No.: PCT/KR2023/003391

§ 371 Date: Mar. 26, 2025

(30) Foreign Application Priority Data
Sep. 27, 2022 (KR) 10-2022-0122616

(51) Int. Cl. C07D 263/58; A61K 31/423; A61P 29/00

(52) U.S. Cl. CPC C07D 263/58; A61K 31/423; A61P 29/00

(57) ABSTRACT

The present disclosure relates to novel benzoxazole derivatives comprising 4-amino-butanamide and anti-inflammatory use thereof.

### TECHNICAL FIELD

The present disclosure relates to novel benzoxazole derivatives comprising 4-amino-butanamide and anti-inflammatory use thereof.

### BACKGROUND ART

Inflammation is an essential immune response of a host upon exposure to tissues and organs to harmful stimuli, such as microbial pathogens, irritants, or toxic cellular components, resulting in ultimate restoration of tissue structure and function. The innate and adaptive immune responses are two major essential components of the host defense system that are important for pathogen-specific defense and immunological memory. In the innate immune system, many types of pathogens are recognized by common molecular patterns. There are a variety of pathogen-associated molecular patterns (PAMPs), including lipopolysaccharides (LPS), aldehyde-derivatized proteins, mannans, teichoic acids, denatured DNA, and bacterial DNA.

Lipopolysaccharide (LPS) is the major component of the cell wall of Gram-negative bacteria. Since it stimulates the release of inflammatory cytokines from various types of cells, bacterial LPS has been widely used to build inflammatory models. LPS is extracted from the bacterial membrane by LPS-binding protein (LPB) in the serum. LPS is then transferred to CD14 by LPB, cleaved into monomeric molecules, and presented to the TLR4-MD-2 complex. The binding and aggregation of LPS with the TLR4-MD-2 complex lead to the activation of multiple signaling components, such as NK-κB and IRF3. Subsequently, various pro-inflammatory cytokines are produced, causing an inflammatory response.

An inflammatory response is induced by LPS, which secretes inflammatory cytokines. The representative inflammatory cytokines include IL-1β, IL-6, and TNF-α. IL-1β has a stimulatory effect on CD4+ T cells and helps them differentiate into T helper cells. IL-1β is synthesized by various types of cells, such as monocytes, macrophages, neutrophils, hepatocytes, and tissue macrophages. IL-6 is involved in hematopoiesis, the final maturation of B-cells into antibody-producing plasma cells, and T cell activation and differentiation. IL-6 is expressed by mononuclear phagocytes, T cells, B cells, fibroblasts, endothelial cells, keratinocytes, hepatocytes, and bone marrow cells. TNF-α is involved in promoting the proliferation of normal cells, exhibits cytolytic or cytostatic activity against tumor cells, and induces inflammatory, antiviral, and immunomodulatory effects. TNF-α is mainly secreted from activated macrophages.

Many small-molecule compounds have been developed to treat immune diseases by modulating cytokine function. These compounds act on proteins involved in the cytokine production pathway to modulate cytokine production and signaling. Representative inflammatory cytokine inhibitors currently under development include tofacitinib, GNE-7915, GSKJ4, VX-765, and ONX 0914, etc. Unlike biopharmaceuticals, such as protein-based therapies, these small-molecule modulators can act on intracellular proteins to modulate abnormal cytokine signaling or downstream sequences.

The present inventors synthesized various benzoxazole derivatives and studied their biological activities, especially anti-inflammatory effects, based on previous studies. The present inventors discovered compounds having a 5-LOX (5-lipoxygenase) inhibitory effect. It was confirmed that these compounds showed an improving effect on methacholine-induced airway hypersensitivity and have potential as a treatment for asthma. Additionally, an inhibitor of IL-6-mediated STAT3 phosphorylation was developed. These compounds also exhibited the effect of suppressing inflammatory cytokine secretion from effector Th1, Th2, and Th17 cells. From these results, it was confirmed that these compounds are potential candidates for the treatment of rheumatoid arthritis (RA). Furthermore, the present inventors discovered compounds that exhibit an inhibitory effect on the expression of IL-1β, IL-6, IL-13, TNF-α, perilipin (PLIN) 2, and PLIN 3 from bone marrow-derived mast cells (BMMC) that are activated by LPS.

### DISCLOSURE

### Technical Problem

An object of the present disclosure is to provide novel benzoxazole derivatives comprising 4-amino-butanamide or pharmaceutically acceptable salts thereof.

Another object of the present disclosure is to provide a method for preparing novel benzoxazole derivatives comprising 4-amino-butanamide or pharmaceutically acceptable salts thereof.

Still another object of the present disclosure is to provide a pharmaceutical composition for preventing or treating an inflammatory disease, comprising novel benzoxazole derivatives comprising 4-amino-butanamide or pharmaceutically acceptable salts thereof, as an active ingredient.

Still another object of the present disclosure is to provide a method for treating an inflammatory disease, comprising administering a pharmaceutical composition for preventing or treating an inflammatory disease, which comprises novel benzoxazole derivatives comprising 4-amino-butanamide or pharmaceutically acceptable salts thereof as an active ingredient, to a subject in need thereof.

### Technical Solution

The present inventors have made extensive efforts to discover small molecule compounds having preventive or therapeutic effects on inflammatory diseases, and as a result, have confirmed that a series of benzoxazole derivatives comprising 4-amino-butanamide are useful for the prevention or treatment of inflammatory diseases by inhibiting the expression levels of representative inflammatory cytokines, such as IL-6, IL-1β, and/or TNF-α, thereby completing the present disclosure.

### Advantageous Effects

The compounds of the present disclosure can effectively inhibit the expression of representative inflammatory cytokines, such as IL-1β, IL-6, and TNF-α, in vitro and in vivo, and therefore can be effectively used for the prevention or treatment of inflammatory diseases.

### BRIEF DESCRIPTION OF THE DRAWINGS

FIG. 1 is a diagram showing the normalized IL-6 mRNA expression levels according to the concentration of Compounds 4d, 5c, 5d, 5f, and 5m.

FIG. 2 is a diagram showing the inhibitory effect of Compounds 5f and 4d on STAT3, IκB, and NF-κB protein levels in AML-12 cells treated with LPS. (a) shows the expression levels of inflammation-related proteins in vitro, and (b) to (d) show the protein expression levels of STAT3, IκB, and NF-κB normalized to the indicated proteins, respectively. As a positive control, cells treated with PBS (vehicle) instead of Compounds 5f and 4d were used, and as a negative control, cells treated only with PBS without LPS were used. Data are expressed as mean±SD (*: p<0.05 compared with the group treated with PBS, +: p<0.05 compared with the group treated with LPS).

FIG. 3 shows the mRNA expression levels of inflammatory cytokines after treatment with Compounds 5f and 4d. (a) to (c) show the mRNA expression levels of IL-1β, IL-6, and TNF-α, respectively. As a positive control, cells treated with PBS (vehicle) instead of Compounds 5f and 4d were used, and as a negative control, cells treated only with PBS without LPS were used. Data are expressed as mean±SD (*: p<0.05 compared with the group treated with PBS, +: p<0.05 compared with the group treated with LPS).

FIG. 4 shows the protective effects of Compounds 5f and 4d in an LPS-induced liver inflammatory disease model in vivo. (a) and (b) show the results of histopathological examination using H&E staining and immunohistochemical staining using F4/80 antibody, respectively. The magnification is ×400, and the F4/80 positive area was quantified. (c) and (d) show the effects of the compounds on serum ALT and AST levels in liver inflammation. Data are expressed as mean±SD (*: p<0.05 compared with the group treated with PBS, +: p<0.05 compared with the group treated with LPS).

### DETAILED DESCRIPTION OF PREFERRED EMBODIMENTS

The present disclosure will be described in detail as follows. Meanwhile, each description and embodiment disclosed in this disclosure may also be applied to other descriptions and embodiments. That is, all combinations of various elements disclosed in this disclosure fall within the scope of the present disclosure. Further, the scope of the present disclosure is not limited by the specific description described below.

Additionally, those of ordinary skill in the art may be able to recognize or confirm, using only conventional experimentation, many equivalents to the particular aspects of the invention described herein. Furthermore, it is also intended that these equivalents be included in the present disclosure.

Further, throughout the whole specification, when a certain portion “includes” or “comprises” a certain component, this indicates that other components are not excluded and may be further included unless explicitly described to the contrary.

Hereinbelow, the present disclosure will be described in detail.

A first aspect of the present disclosure to achieve the objects above provides a compound represented by Formula 1 below or a pharmaceutically acceptable salt thereof:

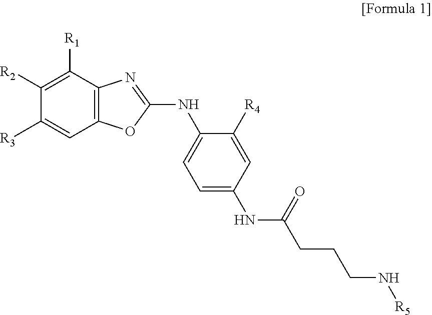

In Formula 1,
R₁ is hydrogen or C₁₋₆ alkyl;
R₂ is hydrogen, halogen, C₁₋₆ alkyl, or C₁₋₆ alkoxy;
R₃ is hydrogen or C₁₋₆ alkyl;
R₄ is hydrogen or C₁₋₆ alkoxy; and
R₅ is hydrogen or C₁₋₆ alkoxycarbonyl.

For example, in Formula 1, R₁ may be hydrogen or methyl; R₂ may be hydrogen, fluoro, chloro, methyl, tert-butyl, or methoxy; R₃ may be hydrogen or methyl; R₄ may be hydrogen or methoxy; and R₅ may be hydrogen or tert-butoxycarbonyl, but is not limited thereto.

Specifically, the compound may be
1. tert-butyl 4-(4-(benzo[d]oxazol-2-ylamino)phenylamino)-4-oxobutylcarbamate;
2. tert-butyl 4-(4-(5-fluorobenzo[d]oxazol-2-ylamino)phenylamino)-4-oxobutylcarbamate;
3. tert-butyl 4-(4-(4-methylbenzo[d]oxazol-2-ylamino)phenylamino)-4-oxobutylcarbamate;
4. tert-butyl 4-(4-(5-methylbenzo[d]oxazol-2-ylamino)phenylamino)-4-oxobutylcarbamate;
5. tert-butyl 4-(4-(6-methylbenzo[d]oxazol-2-ylamino)phenylamino)-4-oxobutylcarbamate;
6. tert-butyl 4-(4-(5-tert-butylbenzo[d]oxazol-2-ylamino)phenylamino)-4-oxobutylcarbamate;
7. tert-butyl 4-(4-(5-methoxybenzo[d]oxazol-2-ylamino)phenylamino)-4-oxobutylcarbamate;
8. tert-butyl 4-(4-(benzo[d]oxazol-2-ylamino)-3-methoxyphenylamino)-4-oxobutylcarbamate;
9. tert-butyl 4-(4-(5-fluorobenzo[d]oxazol-2-ylamino)-3-methoxyphenylamino)-4-oxobutylcarbamate;
10. tert-butyl 4-(4-(5-chlorobenzo[d]oxazol-2-ylamino)-3-methoxyphenylamino)-4-oxobutylcarbamate;
11. tert-butyl 4-(3-methoxy-4-(5-methylbenzo[d]oxazol-2-ylamino)phenylamino)-4-oxobutylcarbamate;
12. tert-butyl 4-(3-methoxy-4-(6-methylbenzo[d]oxazol-2-ylamino)phenylamino)-4-oxobutylcarbamate;
13. tert-butyl 4-(4-(5-tert-butylbenzo[d]oxazol-2-ylamino)-3-methoxyphenylamino)-4-oxobutylcarbamate;
14. 4-amino-N-(4-(benzo[d]oxazol-2-ylamino)phenyl)butanamide;
15. 4-amino-N-(4-(5-fluorobenzo[d]oxazol-2-ylamino)phenyl)butanamide;
16. 4-amino-N-(4-(4-methylbenzo[d]oxazol-2-ylamino)phenyl)butanamide;
17. 4-amino-N-(4-(5-methylbenzo[d]oxazol-2-ylamino)phenyl)butanamide;
18. 4-amino-N-(4-(6-methylbenzo[d]oxazol-2-ylamino)phenyl)butanamide;
19. 4-amino-N-(4-(5-tert-butylbenzo[d]oxazol-2-ylamino)phenyl)butanamide;
20. 4-amino-N-(4-(5-methoxybenzo[d]oxazol-2-ylamino)phenyl)butanamide;
21. 4-amino-N-(4-(benzo[d]oxazol-2-ylamino)-3-methoxyphenyl)butanamide;
22. 4-amino-N-(4-(5-fluorobenzo[d]oxazol-2-ylamino)-3-methoxyphenyl)butanamide;
23. 4-amino-N-(4-(5-chlorobenzo[d]oxazol-2-ylamino)-3-methoxyphenyl)butanamide;
24. 4-amino-N-(3-methoxy-4-(5-methylbenzo[d]oxazol-2-ylamino)phenyl)butanamide;
25. 4-amino-N-(3-methoxy-4-(6-methylbenzo[d]oxazol-2-ylamino)phenyl)butanamide; or
26. 4-amino-N-(4-(5-tert-butylbenzo[d]oxazol-2-ylamino)-3-methoxyphenyl)butanamide, but is not limited thereto.

For example, the compound of the present disclosure may exist in the form of a pharmaceutically acceptable salt. For the salt, acid addition salts formed by pharmaceutically acceptable free acids may be useful. As used herein, the term “pharmaceutically acceptable salt” refers to any organic or inorganic addition salt of the compound, which has a concentration for exhibiting an effective action, is relatively non-toxic and un-harmful to patients, and whose adverse effects resulting from the salt do not deteriorate the advantageous effects of the compound represented by Formula 1.

Acid addition salts may be prepared by a conventional method, for example, by dissolving the compound in an excess amount of aqueous acid solution, followed by precipitating the salt in a water-miscible organic solvent, e.g., methanol, ethanol, acetone, or acetonitrile. An equimolar amount of a compound and an acid or alcohol in water (e.g., glycol monomethylether) may be heated and then the mixture may be dried by evaporation, or the precipitated salt may be subjected to suction filtration.

In particular, organic acids and inorganic acids may be used as a free acid. Examples of inorganic acids may include hydrochloric acid, phosphoric acid, sulfuric acid, nitric acid, tartaric acid, etc.; and examples of organic acids may include methanesulfonic acid, p-toluenesulfonic acid, acetic acid, trifluoroacetic acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid, etc., but are not limited thereto.

Additionally, a pharmaceutically acceptable metal salt may be prepared using a base. An alkali metal salt or alkali earth metal salt may be prepared, for example, by dissolving a compound in an excess amount of an alkali metal hydroxide or alkali earth metal hydroxide solution, filtering a non-dissolved compound salt obtained therefrom, and evaporating the filtrate, followed by drying. In particular, examples of a pharmaceutically acceptable metal salt to be prepared may include sodium, potassium, or calcium salts, but are not limited thereto. Additionally, a corresponding silver salt may be prepared by reacting an alkali metal or alkali earth metal salt with an appropriate silver salt (e.g., silver nitrate), but the preparation method is not limited thereto.

The pharmaceutically acceptable salt of the compound of the present disclosure may include salts of an acidic or basic group that can be present in the compound of Formula 1, unless indicated otherwise. For example, the pharmaceutically acceptable salt may include sodium, calcium, or potassium salts of a hydroxyl group, etc., and other pharmaceutically acceptable salts of an amino group may include hydrobromide, sulfate, hydrogen sulfate, phosphate, hydrogen phosphate, dihydrogen phosphate, acetate, succinate, citrate, tartrate, lactate, mandelate, methanesulfonate (mesylate), p-toluenesulfonate (tosylate), etc., and they can be prepared by a preparation method known in the art.

As a salt of novel benzoxazole derivatives comprising 4-amino-butanamide of the present disclosure, which is a pharmaceutically acceptable salt, any salt of novel benzoxazole derivatives comprising 4-amino-butanamide which exhibits the same pharmacological activity as novel benzoxazole derivatives comprising 4-amino-butanamide can be used without limitation.

Additionally, the compound represented by Formula 1 according to the present disclosure includes not only pharmaceutically acceptable salts thereof, but also solvates such as hydrates that can be prepared therefrom, and all possible stereoisomers, without limitation. The solvate and stereoisomer of the compound represented by Formula 1 may be prepared from the compound represented by Formula 1 using common methods known in the art.

Further, the compound represented by Formula 1 according to the present disclosure may be prepared either in a crystalline form or in a non-crystalline form, and when the compound is prepared in a crystalline form, it may be optionally hydrated or solvated. In the present disclosure, the compound represented by Formula 1 may not only include a stoichiometric hydrate, but also include a compound containing various amounts of water. The solvate of the compound represented by Formula 1 according to the present disclosure includes both stoichiometric solvates and non-stoichiometric solvates.

A second aspect of the present disclosure provides a method for preparing the compound of the first aspect or a pharmaceutically acceptable salt thereof, including:
a first step of reacting a compound represented by Formula 2 below with tert-butyl 4-amino-4-oxobutylcarbamate to prepare a compound represented by Formula 3 below, and
a second step of optionally hydrolyzing in the presence of an acid to remove the tert-butoxycarbonyl protecting group:

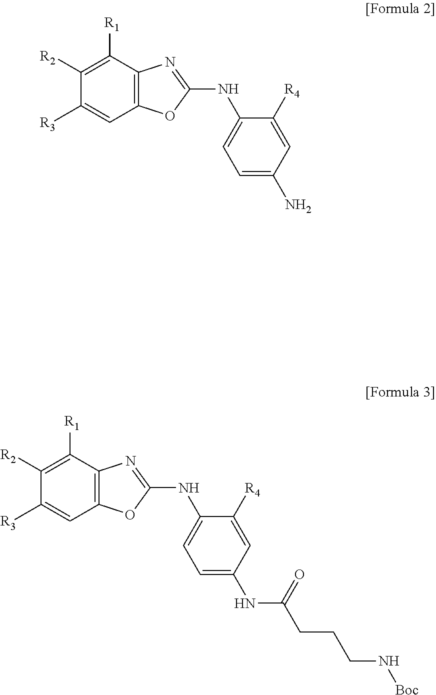

In the Formula 2 or 3,
R₁ to R₄ are as defined in the first aspect.

As used herein, the term “pharmaceutically acceptable salt” is as described above.

For example, the first step may be performed in the presence of a peptide coupling reagent and Hunig's base, but is not limited thereto. For example, the peptide coupling reagent may be benzotriazol-1-yloxytripyrrolidinophosphonium hexafluorophosphate (PyBOP), and the Hunig's base may be diisopropylethylamine, but is not limited thereto. Furthermore, the reaction may be performed while stirring at a low temperature of −5 to 10° C., but is not limited thereto.

For example, the compound represented by Formula 2 above may be commercially purchased and used, or may be prepared according to a series of methods known in the art or using methods appropriately modified therefrom, but is not limited thereto. Specifically, the compound of Formula 2 above may be synthesized in a similar manner to the method disclosed in Patent Application No. 10-2021-0187391, which is an inventor's prior art, and used, but is not limited thereto.

A third aspect of the present disclosure provides a pharmaceutical composition for preventing or treating an inflammatory disease, comprising a compound represented by Formula 1 or a pharmaceutically acceptable salt thereof, as an active ingredient.

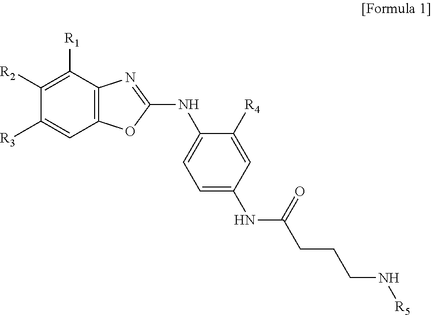

In Formula 1,
R₁ is hydrogen or C₁₋₆ alkyl;
R₂ is hydrogen, halogen, C₁₋₆ alkyl, or C₁₋₆ alkoxy;
R₃ is hydrogen;
R₄ is hydrogen or C₁₋₆ alkoxy; and
R₅ is hydrogen or C₁₋₆ alkoxycarbonyl.

As used herein, the term “pharmaceutically acceptable salt” may be as described above.

For example, In Formula 1, R₁ may be hydrogen or methyl; R₂ may be hydrogen, fluoro, chloro, methyl, tert-butyl, or methoxy; R₃ may be hydrogen; R₄ may be hydrogen or methoxy; and R₅ may be hydrogen or tert-butoxycarbonyl, but is not limited thereto.

Specifically, the compound may be
1. tert-butyl 4-(4-(benzo[d]oxazol-2-ylamino)phenylamino)-4-oxobutylcarbamate;
2. tert-butyl 4-(4-(5-fluorobenzo[d]oxazol-2-ylamino)phenylamino)-4-oxobutylcarbamate;
3. tert-butyl 4-(4-(4-methylbenzo[d]oxazol-2-ylamino)phenylamino)-4-oxobutylcarbamate;
4. tert-butyl 4-(4-(5-methylbenzo[d]oxazol-2-ylamino)phenylamino)-4-oxobutylcarbamate;
5. tert-butyl 4-(4-(5-tert-butylbenzo[d]oxazol-2-ylamino)phenylamino)-4-oxobutylcarbamate;
6. tert-butyl 4-(4-(5-methoxybenzo[d]oxazol-2-ylamino)phenylamino)-4-oxobutylcarbamate;
7. tert-butyl 4-(4-(benzo[d]oxazol-2-ylamino)-3-methoxyphenylamino)-4-oxobutylcarbamate;
8. tert-butyl 4-(4-(5-fluorobenzo[d]oxazol-2-ylamino)-3-methoxyphenylamino)-4-oxobutylcarbamate;
9. tert-butyl 4-(4-(5-chlorobenzo[d]oxazol-2-ylamino)-3-methoxyphenylamino)-4-oxobutylcarbamate;
10. tert-butyl 4-(3-methoxy-4-(5-methylbenzo[d]oxazol-2-ylamino)phenylamino)-4-oxobutylcarbamate;
11. tert-butyl 4-(4-(5-tert-butylbenzo[d]oxazol-2-ylamino)-3-methoxyphenylamino)-4-oxobutylcarbamate;
12. 4-amino-N-(4-(benzo[d]oxazol-2-ylamino)phenyl)butanamide;
13. 4-amino-N-(4-(5-fluorobenzo[d]oxazol-2-ylamino)phenyl)butanamide;
14. 4-amino-N-(4-(4-methylbenzo[d]oxazol-2-ylamino)phenyl)butanamide;
15. 4-amino-N-(4-(5-methylbenzo[d]oxazol-2-ylamino)phenyl)butanamide;
16. 4-amino-N-(4-(5-tert-butylbenzo[d]oxazol-2-ylamino)phenyl)butanamide;
17. 4-amino-N-(4-(5-methoxybenzo[d]oxazol-2-ylamino)phenyl)butanamide;
18. 4-amino-N-(4-(benzo[d]oxazol-2-ylamino)-3-methoxyphenyl)butanamide;
19. 4-amino-N-(4-(5-fluorobenzo[d]oxazol-2-ylamino)-3-methoxyphenyl)butanamide;
20. 4-amino-N-(4-(5-chlorobenzo[d]oxazol-2-ylamino)-3-methoxyphenyl)butanamide;
21. 4-amino-N-(3-methoxy-4-(5-methylbenzo[d]oxazol-2-ylamino)phenyl)butanamide; or
22. 4-amino-N-(4-(5-tert-butylbenzo[d]oxazol-2-ylamino)-3-methoxyphenyl)butanamide, but is not limited thereto.

As used herein, the term “prevention” refers to all actions to inhibit or delay the occurrence, spread, or recurrence of inflammatory diseases by administration of the composition of the present disclosure, and the term “treatment” refers to all actions to improve or beneficially change the symptoms of the above disease by administration of the composition of the present disclosure.

For example, the composition of the present disclosure may modulate the TLR signaling pathway by reducing the mRNA expression of inflammatory cytokines in vitro or in vivo. The inflammatory cytokine that may be regulated by the composition of the present disclosure may be one or more selected from the group consisting of IL-6, IL-1β, and TNF-α. The compound of Formula 1 contained as an effective ingredient in the composition of the present disclosure may reduce the mRNA expression of representative inflammatory cytokines such as IL-6, IL-1β, and/or TNF-α, and therefore may be used for the prevention or treatment of inflammatory diseases.

For example, the term “inflammatory disease” is a generic term for diseases with inflammation as the main lesion, and may be an acute or chronic inflammatory disease such as infectious disease, allergy, autoimmune disease, or metabolic disease. Specifically, it may include allergic diseases including allergic asthma, allergic rhinitis, allergic mucositis, urticaria, and anaphylaxis; myopathy including systemic sclerosis, dermatomyositis, and inclusion body myositis; arthritis; atopic dermatitis; psoriasis; asthma; multiple sclerosis; ssRNA and dsRNA virus infection; sepsis; multiple chondritis; scleroderma; eczema; gout; periodontal disease; Behcet's syndrome; edema; vasculitis; Kawasaki disease; diabetic retinitis; autoimmune pancreatitis; vasculitis; glomerulonephritis; acute and chronic bronchitis; Crohn's disease; and influenza infection, but the diseases are not limited thereto.

However, diseases that may be prevented or treated using the pharmaceutical composition of the present disclosure are not limited thereto, and any disease that can exhibit a preventive or therapeutic effect by reducing the expression of mRNA of IL-6, IL-1β, and/or TNF-α, etc., may be included in the scope of the present disclosure.

Preferably, the pharmaceutical composition according to the present disclosure may contain a compound represented by Formula 1 or a pharmaceutically acceptable salt thereof, as an active ingredient, in an amount of 0.1 to 75 wt %, more preferably 1 to 50 wt %, based on the total weight of the composition.

The composition of the present disclosure may further include a pharmaceutically acceptable carrier, a diluent, or an excipient, and may be formulated and used in various forms including oral formulations such as powders, granules, tablets, capsules, suspensions, emulsions, syrups, aerosols, etc., and injectable drugs such as sterile injection solutions, etc., according to a general method for each purpose of use, and may be administered orally, or through various routes including intravenous, intraperitoneal, subcutaneous, rectal, topical administrations, etc. Examples of suitable carriers, excipients, or diluents that can be included in the composition may include lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methylhydroxybenzoate, propyl hydroxybenzoate, talc, magnesium stearate, and mineral oil. Further, the composition of the present disclosure may further include a filler, an anti-aggregating agent, a lubricant, a wetting agent, a flavoring agent, an emulsifier, a preservative, etc.

Solid preparations for oral administration include tablets, pills, powders, granules, capsules, etc., and such a solid preparation is formulated by mixing one or more excipients, such as starch, calcium carbonate, sucrose, lactose, gelatin, etc. with the composition. Further, in addition to a simple excipient, a lubricant such as magnesium stearate or talc may be used.

Liquid preparations for oral administration include a suspension, a solution for internal use, an emulsion, a syrup, etc., and may include various excipients, such as a wetting agent, a sweetening agent, a fragrance, a preservative, etc., in addition to water and liquid paraffin, which are commonly used as a simple diluent.

Preparations for parenteral administration include an aqueous solvent, a non-aqueous solvent, a suspension agent, an emulsifier, a lyophilized preparation, and a suppository, which are sterilized. As the non-aqueous solvent or the suspension agent, propylene glycol, polyethylene glycol, vegetable oil such as olive oil, injectable ester such as ethyl oleate, etc., may be used. As a base of the suppository, witepsol, macrogol, twin 61, cacao butter, laurin butter, glycerogelatin, etc. may be used. Meanwhile, injection drugs may include conventional additives such as a solubilizing agent, an isotonic agent, a suspension agent, an emulsifier, a stabilizing agent, a preservative, etc.

The formulation may be prepared by a conventional mixing, granulating, or coating method, and may contain an active ingredient in an amount of about 0.1 wt % to 75 wt %, preferably about 0.1 wt % to 50 wt %. The unit formulation for a mammal weighing about 50 kg to 70 kg contains about 10 mg to 200 mg of an active ingredient.

In particular, the composition of the present disclosure is administered in a pharmaceutically effective amount. As used herein, the term “pharmaceutically effective amount” refers to an amount sufficient to treat a disease at a reasonable benefit/risk ratio applicable to medical treatment and not cause side effects, and the level of the effective amount may be determined depending on the patient's health status, type of disease, severity, activity of drug, sensitivity to drug, administration method, administration time, administration route, excretion rate, treatment period, factors including drugs used in combination or concurrently, and other factors well known in the medical field. The composition of the present disclosure may be administered as an individual therapeutic agent or administered in combination with other therapeutic agents, may be administered sequentially or simultaneously with a conventional therapeutic agent, and may be administered in a single dose or multiple doses. It is important to administer an amount that can achieve the maximum effect with a minimum amount without adverse effects in consideration of all of the above factors, which may be easily determined by those skilled in the art.

For example, since the administration dose may increase or decrease depending on the administration route, disease severity, sex, weight, age, etc., the administration does not limit the scope of the present disclosure in any way.

Specifically, the effective amount of the compound in the composition of the present disclosure may vary depending on the patient's age, sex, and weight, and is generally administered daily or every other day, or may be divided into 1 to 3 times a day in an amount of 1 to 100 mg, preferably 5 to 60 mg 1 to 100 mg per kg of body weight, preferably 5 to 60 mg per kg. However, since the effective amount may increase or decrease depending on the administration route, severity of the disease, sex, weight, age, etc., the above dose does not limit the scope of the present disclosure in any way.

A fourth aspect of the present disclosure provides a method for treating an inflammatory disease, comprising administering the pharmaceutical composition of the third aspect to a subject in need thereof.

As used herein, the terms “pharmaceutical composition of the third aspect” and “inflammatory disease” are as described above.

As used herein, the term “subject” refers to any animal including monkeys, cows, horses, sheep, pigs, chickens, turkeys, quails, cats, dogs, mice, rats, rabbits, or guinea pigs in addition to humans, which have developed or may develop inflammatory disease, and the subject disease may be effectively prevented or treated by administering the pharmaceutical composition of the present disclosure to the subject. In addition, the pharmaceutical composition of the present disclosure may be administered in combination with a conventional therapeutic agent.

As used herein, the term “administration” refers to providing a predetermined substance to a patient by any suitable method, and the administration route of the composition of the present disclosure may be achieved through any general route as long as it can reach target tissues. The composition may be administered through intraperitoneal administration, intravenous administration, intramuscular administration, subcutaneous administration, intradermal administration, oral administration, topical administration, intranasal administration, intrapulmonary administration, or rectal administration, but the administration route is not limited thereto. Additionally, the pharmaceutical composition of the present disclosure may be administered by any device capable of delivering an active substance to a target cell. Preferred administration routes and formulations include intravenous injection drugs, subcutaneous injection drugs, intradermal injection drugs, intramuscular injection drugs, dropwise injection drugs, etc. The injection drugs may be prepared using an aqueous solvent such as a physiological saline solution, Ringer's solution, etc., or a non-aqueous solvent such as vegetable oils, higher fatty acid esters (e.g., ethyl oleate, etc.), and alcohols (e.g., ethanol, benzyl alcohol, propylene glycol, glycerin, etc.), and may include a pharmaceutical carrier such as a stabilizer for preventing denaturation (e.g., ascorbic acid, sodium bisulfite, sodium pyrosulfite, BHA, tocopherol, EDTA, etc.), an emulsifier, a buffer for pH control, and a preservative for inhibiting microbial growth (e.g., phenylmercuric nitrate, thiomersal, benzalkonium chloride, phenol, cresol, benzyl alcohol, etc.).

The term “therapeutically effective amount” used in combination with an active ingredient in the present disclosure means an amount of novel benzoxazole derivatives comprising 4-amino-butanamide or a pharmaceutically acceptable salt thereof, which is effective in preventing or treating a target disease.

The pharmaceutical composition of the present disclosure may further include, depending on the type of diseases to be prevented or treated, a known drug used for the prevention or treatment of each disease, in addition to the novel benzoxazole derivatives comprising 4-amino-butanamide or a pharmaceutically acceptable salt thereof, as an active ingredient. For example, for the prevention or treatment of cancer, the pharmaceutical composition may further include a known drug in addition to the novel benzoxazole derivatives comprising 4-amino-butanamide or a pharmaceutically acceptable salt thereof, as an active ingredient, and may be used in combination with other known treatments for the treatment of these diseases.

### MODE FOR CARRYING OUT THE INVENTION

Hereinafter, the present disclosure will be described in more detail through exemplary embodiments. However, these exemplary embodiments are for illustrative purposes only and are not intended to limit the scope of the present disclosure.

### Materials and Methods

Melting points were measured without calibration on an electrothermal digital melting point apparatus (Buchi, Germany). ¹H-NMR spectra were recorded on the Varian NMR AS and Varian Unity Inova 400 MHz NMR spectrometers. Chemical shifts were expressed in parts per million (ppm, d) relative to the solvent peaks. ¹H NMR data were expressed as peak multiplicities (s for singlet; d for doublet; t for triplet; q for quartet; and m for multiplet). Coupling constants were expressed in hertz (Hz). MS spectra were measured using the Jeol JMS 700 high-resolution mass spectrometer at the Korea Basic Science Institute (Daegu). Reagents were purchased in commercial grade from Sigma-Aldrich Co. (St. Louis, MO, USA), Merck (Darmstadt, Germany), and Duksan Pure Chemical Co. (Ansan, Korea).

### Example 1: Synthesis of 1-(Substituted-2-hydroxyphenyl)-3-(4-nitrophenyl)thiourea Derivatives (1a-g)

First, 25 mL of methanol was added to variously substituted 2-aminophenols (100 mg, 1 eq.), and 1-isothiocyanato-4-nitrobenzene or 1-isothiocyanato-2-methoxy-4-nitrobenzene (1 eq.). The reaction mixture was stirred at room temperature for 24 hours. After the reaction was completed, the organic solvent was removed from the reaction mixture under reduced pressure to obtain a series of 1-(substituted-2-hydroxyphenyl)-3-(4-nitrophenyl)thioureas or 1-(2-hydroxyphenyl)-3-(2-methoxy-4-nitrophenyl)thioureas (1a-m).

### 1-(2-hydroxyphenyl)-3-(4-nitrophenyl)thiourea (1a)

Yellow solid (95.9%),

mp 148-150° C.,

¹H-NMR (DMSO-d₆, 400 MHz) δ 8.30 (dd, J=7.2, 2.0 Hz, 1H), 8.25 (dd, J=7.2, 2.0 Hz, 1H), 7.90 (brs, 1H), 7.83 (dd, J=7.2, 2.0 Hz, 1H), 7.70 (d, J=9.2 Hz, 1H), 7.30 (d, J=7.2 Hz, 1H), 7.09 (dd, J=9.2, 1.2 Hz, 1H), 7.03 (td, J=7.9, 1.2 Hz, 1H),

HR-FABMS Calcd. for C₁₃H₁₂N₃O₃S (M⁺+H): 290.0601, Found: 290.0592.

### 1-(5-fluoro-2-hydroxyphenyl)-3-(4-nitrophenyl)thiourea (1b)

Green-brown solid (78.0%),

mp 139-141° C.,

¹H-NMR (DMSO-d₆, 400 MHz) δ 10.71 (s, 1H), 10.04 (s, 1H), 9.59 (s, 1H), 8.22 (dt, J=9.2, 2.4 Hz, 2H), 7.98-7.94 (m, 3H) 6.89-6.85 (m, 2H),

HR-FABMS Calcd. for C₁₃H₁₁FN₃O₃S (M⁺+H): 308.0500, Found: 308.0503.

### 1-(2-hydroxy-6-methylphenyl)-3-(4-nitrophenyl)thiourea (1c)

Yellow solid (91.3%),

mp 168-170° C.,

¹H-NMR (CDCl₃, 400 MHz) δ 8.23 (dd, J=9.0, 2.8 Hz, 2H), 7.83 (dd, J=7.2, 2.4 Hz, 2H), 7.16 (t, J=3.2, 2.4 Hz, 2H), 6.94 (t, J=8.0, 7.2 Hz, 1H),

HR-FABMS Calcd. for C₁₄H₁₄N₃O₃S (M⁺+H): 304.3443, Found: 304.0751.

### 1-(2-hydroxy-5-methylphenyl)-3-(4-nitrophenyl)thiourea (1d)

Yellow solid (85.8%),

mp 158-159° C.,

¹H-NMR (CDCl₃, 400 MHz) δ 8.25 (dt, J=9.4, 2.6 Hz, 2H), 7.86 (brs, 1H), 7.73 (brs, 1H), 7.69 (d, J=9.2 Hz, 2H), 7.11 (dd, J=8.2, 1.8 Hz, 1H), 7.07 (s, 1H), 6.98 (d, J=8.4 Hz, 1H), 2.31 (s, 3H),

HR-FABMS Calcd. for C₁₄H₁₄N₃O₃S (M⁺+H): 304.3443, Found: 304.0752.

### 1-(2-hydroxy-4-methylphenyl)-3-(4-nitrophenyl)thiourea (1e)

Mustard solid (91.3%),

mp 130-136° C.,

¹H-NMR (CDCl₃, 400 MHz) δ 8.23 (dd, J=7.2, 2.0 Hz, 2H), 7.83 (brs, 1H), 7.71 (d, J=9.2 Hz, 2H), 7.15 (d, J=8.0 Hz, 1H), 6.90 (s, 1H), 6.83 (d, J=8.0 Hz, 1H), 2.36 (s, 3H),

HR-FABMS Calcd. for C₁₄H₁₄N₃O₃S (M⁺+H): 304.3443, Found: 304.0749.

### 1-(5-tert-butyl-2-hydroxyphenyl)-3-(4-nitrophenyl)thiourea (1f)

Pale grey-yellow solid (71.8%),

mp 141-145° C.,

¹H-NMR (CDCl₃, 400 MHz) δ 8.25 (d, J=8.8 Hz, 2H), 7.92 (brs, 1H), 7.80 (brs, 1H), 7.70 (d, J=9.2 Hz, 2H), 7.33 (dd, J=8.8, 2.4 Hz, 1H), 7.25 (d, J=2.4 Hz, 1H), 7.02 (d, J=8.4 Hz, 1H), 1.3 (s, 9H),

HR-FABMS Calcd. for C₁₇H₂₀N₃O₃S (M⁺+H): 346.4240, Found: 346.1221.

### 1-(2-hydroxy-5-methoxyphenyl)-3-(4-nitrophenyl)thiourea (1)

Green powder (89.0%),

mp 122-124° C.,

¹H-NMR (CDCl₃, 400 MHz) δ 8.30-8.22 (m, 3H), 7.85 (s, 1H), 7.74 (s, 1H), 7.67 (d, J=7.2 Hz, 2H), 7.03-7.00 (m, 1H), 6.88-6.86 (m, 2H), 3.78 (s, 3H),

HR-FABMS Calcd. for C₁₄H₁₄N₃O₄S (M⁺+H): 320.07, Found: 320.0699.

### Example 2: Synthesis of 1-(substituted-2-hydroxyphenyl)-3-(2-methoxy-4-nitrophenyl)thiourea Derivatives (1 h-m)

A series of 1-(substituted-2-hydroxyphenyl)-3-(4-nitrophenyl)thioureas were obtained in a similar manner as in Example 1 using 1-isocyanato-2-methoxy-4-nitrobenzene (1 eq.).

### 1-(2-hydroxyphenyl)-3-(2-methoxy-4-nitrophenyl)thiourea (1h)

Bright yellow solid (90.6%),

mp 135-138° C.,

¹H-NMR (CDCl₃, 400 MHz) δ 8.85 (d, J=8.8 Hz, 1H), 8.37 (s, 1H), 7.92 (dd, J=8.8, 2.4 Hz, 1H), 7.71 (d, J=2.4 Hz, 1H), 7.32 (q, J=8.0 Hz, 1H), 7.09 (d, J=7.2 Hz, 1H), 7.04 (td, J=7.7, 1.0 Hz, 1H), 6.04 (brs, 1H), 3.84 (s, 3H),

HR-FABMS Calcd. for C₁₄H₁₄N₃O₄S (M⁺+H): 320.3437, Found: 320.0700.

### 1-(5-fluoro-2-hydroxyphenyl)-3-(2-methoxy-4-nitrophenyl)thiourea (1i)

Yellow solid (99.3%),

mp 133-137° C.,

¹H-NMR (DMSO-d₆, 400 MHz) δ 10.09 (s, 1H), 10.01 (s, 1H), 9.97 (s, 1H), 8.67 (d, J=9.2 Hz, 1H), 7.93-7.84 (m, 3H), 6.89-6.86 (m, 2H), 4.01 (s, 3H),

HR-FABMS Calcd. For C₁₄H₁₃FN₃O₄S (M⁺+H): 338.0605, Found: 338.0606.

### 1-(5-chloro-2-hydroxyphenyl)-3-(2-methoxy-4-nitrophenyl)thiourea (1j)

Yellowish brown solid (86.0%),

mp 153-154° C.,

¹H-NMR (DMSO-d₆, 400 MHz) δ 10.27 (s, 1H), 10.08 (s, 1H), 9.97 (s, 1H), 8.69 (d, J=9.2 Hz, 1H), 8.01 (d, J=2.4 Hz, 1H), 7.89 (dd, J=9.0, 2.6 Hz, 1H), 7.84 (d, J=2.4 Hz, 1H), 7.07 (dd, J=8.6, 2.6 Hz, 1H), 6.92 (d, J=8.4 Hz, 1H), 4.01 (s, 3H),

HR-FABMS Calcd. for C₁₄H₁₂ClN₃O₄S (M⁺+H): 354.0315, Found: 354.0319.

### 1-(2-hydroxy-5-methylphenyl)-3-(2-methoxy-4-nitrophenyl)thiourea (1k)

Yellowish brown solid (94.3%),

mp 131-137° C.,

¹H-NMR (CDCl₃, 400 MHz) δ 8.85 (dd, J=8.8, 4.4 Hz, 1H), 8.36 (brs, 1H), 7.94 (dd, J=8.8, 4.4 Hz, 1H), 7.73 (d, J=2.0 Hz, 1H), 7.60 (brs, 1H), 7.13 (d, J=4.8 Hz, 1H), 6.98 (d, J=8.4 Hz, 1H), 5.72 (brs, 1H), 3.86 (s, 3H), 2.33 (s, 3H),

HR-FABMS Calcd. for C₁₅H₁₆N₃O₄S (M⁺+H): 334.3702, Found: 334.0857.

### 1-(2-hydroxy-4-methylphenyl)-3-(2-methoxy-4-nitrophenyl)thiourea (11)

Yellow solid (88.7%),

mp 140-142° C.,

¹H-NMR (DMSO-d₆, 400 MHz) δ 9.96 (s, 1H), 9.75 (s, 2H), 8.83 (s, 1H), 7.89-7.86 (m, 1H), 7.81 (d, J=2.0 Hz, 1H), 7.47 (s, 1H), 6.73 (s, 1H), 6.62 (d, J=8.0 Hz, 1H), 3.98 (s, 3H), 2.23 (s, 3H),

HR-FABMS Calcd. for C₁₅H₁₆N₃O₄S (M⁺+H): 334,0856, Found: 334.0856.

### 1-(5-tert-butyl-2-hydroxyphenyl)-3-(2-methoxy-4-nitrophenyl)thiourea (1m)

Yellow solid (87.9%),

mp 63-65° C.,

¹H-NMR (DMSO-d₆, 400 MHz) δ 10.03 (s, 1H), 9.68 (s, 2H), 8.81 (d, J=8.8 Hz, 1H), 7.88 (dd, J=8.8, 2.4 Hz, 1H), 7.82 (d, J=2.4 Hz, 1H), 7.74 (s, 1H), 7.08 (dd, J=8.8, 2.4 Hz, 1H), 6.84 (d, J=8.4 Hz, 1H), 3.89 (s, 3H), 1.24 (s, 9H),

HR-FABMS Calcd. for C₁₈H₂₂N₃O₄S (M⁺+H): 376.1326, Found: 376.1326.

### Example 3: Synthesis of Substituted-N-(4-nitrophenyl)benzo[d]oxazol-2-amine Derivatives (2a-g)

First, 5 mL of dry acetonitrile was added to potassium superoxide (KO₂, 5 eq.) under an N₂ atmosphere. The acetonitrile solution of each of the 1-(substituted-2-hydroxyphenyl)-3-(4-nitrophenyl)thiourea derivatives (1a-g, 100 mg, 1 eq.) prepared according to Example 1 was added dropwise to the KO₂-acetonitrile mixture. The reaction mixture was stirred vigorously for 16 hours at room temperature under an N₂ atmosphere. After completion of the reaction, the reaction mixture was added with cold water, extracted with dichloromethane, and washed with saline. The resultant was dried over anhydrous MgSO₄ and filtered, then the organic solvent was removed under reduced pressure to obtain Compounds 2a-g.

### N-(4-nitrophenyl)benzo[d]oxazol-2-amine (2a)

Yellow solid (72.4%),

mp 219-220° C.,

¹H-NMR (CDCl₃, 400 MHz) δ 8.31 (d, J=2.0 Hz, 1H), 8.29 (d, J=2.4 Hz, 1H), 7.83 (dd, J=2.4 Hz, 2H), 7.57 (d, J=7.6 Hz, 1H), 7.41 (d, J=8.0 Hz, 1H), 7.30 (t, J=7.7, 1.2 Hz, 1H), 7.22 (t, J=7.8 Hz, 1H),

HR-FABMS Calcd. for C₁₃H₁₀N₃O₃ (M⁺+H): 256.2368, Found: 256.0717.

### 5-fluoro-N-(4-nitrophenyl)benzo[d]oxazol-2-amine (2b)

Bright yellow solid (63.7%),

mp 254-256° C.,

¹H-NMR (CDCl₃, 400 MHz) δ 8.30 (dt, J=7.0, 2.2 Hz, 2H), 9.82 (dt, J=7.0, 2.2 Hz, 2H), 7.27 (s, 1H), 7.31 (q, J=4.4 Hz, 1H), 6.93 (td, J=9.0, 2.4 Hz, 1H),

HR-FABMS Calcd. for C₁₃H₉FN₃O₃ (M⁺+H): 274.0622, Found: 274.0624.

### 4-methyl-N-(4-nitrophenyl)benzo[d]oxazol-2-amine (2c)

Yellow solid (83.8%),

mp 227-228° C.,

¹H-NMR (CDCl₃, 400 MHz) δ 8.30 (dt, J=9.6, 2.6 Hz, 2H), 7.84 (dt, J=8.4, 5.0 Hz, 2H), 7.23 (t, J=Cl, 4.4 Hz, 1H), 7.11 (d, J=1.2 Hz, 1H), 7.10 (s, 1H), 2.59 (s, 3H),

HR-FABMS Calcd. for C₁₄H₁₂N₃O₃ (M⁺+H): 270.2634, Found: 270.0877.

### 5-methyl-N-(4-nitrophenyl)benzo[d]oxazol-2-amine (2d)

Bright yellow solid (67.4%),

mp 256-257° C.,

¹H-NMR (CDCl₃, 400 MHz) δ 8.29 (dd, J=7.2, 2.0 Hz, 2H), 7.82 (dd, J=6.8, 2.0 Hz, 2H), 7.37 (s, 1H), 7.28 (s, 1H), 7.02 (d, J=8.0 Hz, 1H), 2.45 (s, 3H),

HR-FABMS Calcd. for C₁₄H₁₂N₃O₃ (M⁺+H): 270.2634, Found: 270.0879.

### 6-methyl-N-(4-nitrophenyl)benzo[d]oxazol-2-amine (2e)

Yellow solid (69.0%),

mp 226-228° C.,

¹H-NMR (CDCl₃, 400 MHz) δ 8.29 (dt, J=9.5, 2.7 Hz, 2H), 7.81 (dt, J=9.8, 2.6 Hz, 2H), 7.44 (d, J=8.0 Hz, 1H), 7.22 (s, 1H), 7.11 (d, J=8.4 Hz, 1H), 2.47 (s, 3H),

HR-FABMS Calcd. for C₁₄H₁₂N₃O₃ (M⁺+H): 270.2634, Found: 270.0874.

### 5-tert-butyl-N-(4-nitrophenyl)benzo[d]oxazol-2-amine (2f)

Yellow solid (69.0%),

mp 217-218′C,

¹H-NMR (CDCl₃, 400 MHz) δ 8.29 (dt, J=9.7, 2.7 Hz, 2H), 7.82 (dt, J=9.7, 2.5 Hz, 2H), 7.62 (d, J=2.0 Hz, 1H), 7.31 (d, J=8.8 Hz, 1H), 7.28 (d, J=1.6 Hz, 1H), 1.38 (s, 9H),

HR-FABMS Calcd. for C₁₇H₁₈N₃O₃ (M⁺+H): 312.3431, Found: 312.1343.

### 5-methoxy-N-(4-nitrophenyl)benzo[d]oxazol-2-amine (2q)

Yellow powder (52%),

mp 217-221° C.,

¹H-NMR (CDCl₃, 400 MHz) δ 8.31-8.27 (m, 2H), 7.83-7.79 (m, 2H), 7.39 (s, 1H), 7.11 (d, J=2.8 Hz, 1H), 6.79 (dd, J=8.8, 2.4 Hz, 1H), 6.63 (d, J=9.2 Hz, 1H), 3.86 (s, 3H),

HR-FABMS Calcd. for C₁₄H₁₂N₃O₄ (M⁺+H): 286.0823, Found: 286.0822.

### Example 4: Synthesis of Substituted-N-(2-methoxy-4-nitrophenyl)benzo[d]oxazol-2-amine Derivatives (2h-m)

A series of substituted-N-(4-nitrophenyl)benzo[d]oxazol-2-amine derivatives (2h-m) were obtained in a similar manner as in Example 3 above using the 1-(substituted-2-hydroxyphenyl)-3-(2-methoxy-4-nitrophenyl)thiourea derivatives (1h-m, 1 eq.) prepared according to Example 2 above.

### N-(2-methoxy-4-nitrophenyl)benzo[d]oxazol-2-amine (2h)

Yellow solid (86.9%),

mp 168-169° C.,

¹H-NMR (CDCl₃, 400 MHz) δ 8.68 (d, J=8.8 Hz, 1H), 8.05 (dd, J=9.2, 2.4 Hz, 1H), 7.93 (brs, 1H), 7.81 (d, J=2.4 Hz, 1H), 7.58 (dd, J=7.6, 0.8 Hz, 1H), 7.40 (d, J=8.4 Hz, 1H), 7.25 (dtd, J=33.2, 7.6, 1.2 Hz, 2H), 4.07 (s, 3H),

HR-FABMS Calcd. for C₁₄H₁₂N₃O₄ (M⁺+H): 286.2628, Found: 286.0824.

### 5-fluoro-N-(2-methoxy-4-nitrophenyl)benzo[d]oxazol-2-amine (2i)

Yellow solid (70.4%),

mp 199-201° C.,

¹H-NMR (DMSO-d₆, 400 MHz) δ 10.54 (s, 1H), 8.57 (d, J=8.8 Hz, 1H), 7.99 (d, J=2.4 Hz, 1H), 7.51 (dd, J=8.8, 4.0 Hz, 1H), 7.35 (d, J=8.8 Hz, 1H), 7.01-6.96 (m, 1H), 3.98 (s, 3H),

HR-FABMS Calcd. for C₁₄H₁₁FN₃O₄ (M⁺+H): 304.0728, Found: 304.0728.

### 5-chloro-N-(2-methoxy-4-nitrophenyl)benzo[d]oxazol-2-amine (2)

Yellow solid (85.1%),

mp 202-204° C.,

¹H-NMR (DMSO-d₆, 400 MHz) δ 10.59 (s, 1H), 8.59 (d, J=9.2 Hz, 1H), 8.03 (dd, J=9.2, 2.4 Hz, 1H), 7.85 (d, J=2.4 Hz, 1H), 7.62 (d, J=2.0 Hz, 1H), 7.59 (d, J=8.8 Hz, 1H), 7.24 (dd, J=8.4, 2.0 Hz, 1H), 4.01 (s, 3H),

HR-FABMS Calcd. for C₁₄H₁₀ClN₃O₄ (M+H)⁺: 320.0433, Found: 320.0435.

### N-(2-methoxy-4-nitrophenyl)-5-methylbenzo[d]oxazol-2-amine (2k)

Yellow solid (78.1%),

mp 163-168° C.,

¹H-NMR (CDCl₃, 400 MHz) δ 8.66 (d, J=8.8 Hz, 1H), 8.04 (dd, J=8.8, 2.8 Hz, 1H), 7.89 (brs, 1H), 7.80 (d, J=2.4 Hz, 1H), 7.37 (s, 1H), 7.26 (d, J=8.4 Hz, 1H), 7.01 (dd, J=8.4, 0.8 Hz, 1H), 4.06 (s, 3H), 2.45 (s, 3H),

HR-FABMS Calcd. for C₁₅H₁₄N₃O₄ (M⁺+H): 300.2894, Found: 300.0982.

### N-(2-methoxy-4-nitrophenyl)-6-methylbenzo[d]oxazol-2-amine (21)

Yellow solid (61.9%),

mp 205-207′C,

¹H-NMR (DMSO-d₆, 400 MHz) δ 10.33 (s, 1H), 8.62 (d, J=9.2 Hz, 1H), 8.00 (dd, J=9.2, 2.4 Hz, 1H), 7.79 (d, J=2.4 Hz, 1H), 7.39 (d, J=8.0 Hz, 1H), 7.07 (d, J=8.0 Hz, 1H), 3.99 (s, 3H), 2.40 (s, 3H),

HR-FABMS Calcd. for C₁₅H₁₄N₃O₄ (M⁺+H): 300.0979, Found: 300.0980.

### 5-tert-butyl-N-(2-methoxy-4-nitrophenyl)benzo[d]oxazol-2-amine (2m)

Yellow solid (62.4%),

mp 150-153° C.,

¹H-NMR (DMSO-d₆, 400 MHz) δ 10.35 (s, 1H), 8.64 (d, J=8.8 Hz, 1H), 8.00 (dd, J=9.2, 2.0 Hz, 1H), 7.80 (s, 1H), 7.53 (s, 1H), 7.41 (d, J=7.6 Hz, 1H), 7.22 (d, J=7.2 Hz, 1H), 3.99 (s, 3H), 1.33 (s, 9H),

HR-FABMS Calcd. for C₁₈H₂₀N₃O₄ (M⁺+H): 342.1448, Found: 342.1451.

### Example 5: Synthesis of N¹-(Substituted-benzo[d]oxazol-2-yl)benzene-1,4-diamine Derivatives (3a-g)

10 mL of ethanol was added to the substituted-N-(4-nitrophenyl)benzo[d]oxazol-2-amine derivatives (2a-g, 100 mg, 1 eq.) prepared according to Example 3 above, and Tin(II) chloride (12 eq.) was added. The reaction mixture was sonicated at room temperature for 3 hours. Potassium hydroxide solution was added to the reaction solution, and the mixture was extracted with ethyl acetate and washed with saline. The resultant was dried over anhydrous MgSO₄ and filtered, then the organic solvent was removed under reduced pressure to obtain Compounds 3a-f.

Alternatively, the substituted-N-(4-nitrophenyl)benzo[d]oxazol-2-amine derivatives (2a-g, 100 mg, 1 eq.) prepared according to Example 3 above were dissolved in methanol, and an appropriate amount of Pd/C was added. The mixture was allowed to react by hydrogen substitution while stirring at room temperature for 3 hours, and then filtered to remove Pd/C. Then, the solvent was removed by evaporation under reduced pressure to obtain Compounds 3a-f.

### N¹-(benzo[d]oxazol-2-yl)benzene-1,4-diamine (3a)

Dark grey solid (40.9%),

mp 198-201° C.,

¹H-NMR (DMSO-d₆, 400 MHz) δ 10.02 (brs, 1H), 7.39 (d, J=7.6 Hz, 1H), 7.36 (dd, J=6.8, 2.0 Hz, 2H), 7.33 (s, 1H), 7.15 (td, J=7.6, 0.8 Hz, 1H), 7.04 (td, J=7.8, 1.2 Hz, 1H), 6.58 (dt, J=9.4, 2.6 Hz, 2H), 4.84 (s, 2H),

HR-FABMS Calcd. for C₁₃H₁₂N₃O (M⁺+H): 226.2539, Found: 226.0975.

### N¹-(5-fluorobenzo[d]oxazol-2-yl)benzene-1,4-diamine (3b)

Dark grey solid (39.3%),

mp 187-191° C.,

¹H-NMR (DMSO-d₆, 400 MHz) δ 10.16 (brs, 1H), 7.39 (q, J=4.3 Hz, 1H), 7.33 (dt, J=9.1, 2.5 Hz, 2H), 7.17 (dd, J=9.4, 2.6 Hz, 1H), 6.84 (td, J=9.3, 2.6 Hz, 1H), 6.57 (dt, J=9.0, 2.4 Hz, 2H), 4.87 (s, 2H),

HR-FABMS Calcd. for C₁₃H₁₁FN₃O (M⁺+H): 244.0881, Found: 244.0885.

### N¹-(4-methylbenzo[d]oxazol-2-yl)benzene-1,4-diamine (3c)

Brown solid (75.2%),

mp 137-138° C.,

¹H-NMR (DMSO-d₆, 400 MHz) δ 9.94 (brs, 1H), 7.36 (dd, J=6.8, 2.0 Hz, 2H), 7.21 (d, J=7.6 Hz, 1H), 6.99-6.91 (m, 2H), 6.59 (d, J=2.0 Hz, 1H), 6.57 (d, J=2.4 Hz, 1H), 4.83 (s, 2H), 2.41 (s, 3H),

HR-FABMS Calcd. for C₁₄H₁₄N₃O (M⁺+H): 240.2805, Found: 240.1140.

### N¹-(5-methylbenzo[d]oxazol-2-yl)benzene-1,4-diamine (3d)

Dark grey solid (64.0%),

mp 170-178° C.,

¹H-NMR (DMSO-d₆, 400 MHz) δ 9.96 (s, 1H), 7.35 (d, J=2.0 Hz, 1H), 7.33 (d, J=1.6 Hz, 1H), 7.26 (d, J=8.4 Hz, 1H), 7.15 (s, 1H), 6.85 (dd, J=8.0, 0.8 Hz, 1H), 6.57 (dt, J=9.6, 2.8 Hz, 2H), 4.85 (brs, 2H), 2.34 (s, 3H),

HR-FABMS Calcd. for C₁₄H₁₄N₃O (M⁺+H): 240.2805, Found: 240.1137.

### N¹-(6-methylbenzo[d]oxazol-2-yl)benzene-1,4-diamine (3e)

Greyish pink solid (49.4%),

mp 153-154° C.,

¹H-NMR (DMSO-d₆, 400 MHz) δ 9.94 (brs, 1H), 7.35 (dt, J=9.2, 2.6 Hz, 2H), 7.22 (d, J=3.2 Hz, 1H), 7.20 (s, 1H), 6.97 (dd, J=8.0, 0.8 Hz, 1H), 6.57 (dt, J=9.0, 2.6 Hz, 2H), 4.82 (s, 2H), 2.36 (s, 3H),

HR-FABMS Calcd. for C₁₄H₁₄N₃O (M⁺+H): 240.2805, Found: 240.1137.

### N¹-(5-tert-butylbenzo[d]oxazol-2-yl)benzene-1,4-diamine (3f)

Grey solid (84.4%),

mp 185-190° C.,

¹H-NMR (DMSO-d₆, 400 MHz) δ 9.96 (s, 1H), 7.36 (dt, J=9.2, 2.8 Hz, 3H), 7.28 (d, J=8.4 Hz, 1H), 7.07 (dd, J=8.4, 2.0 Hz, 1H), 6.57 (dt, J=9.2, 2.6 Hz, 2H), 4.82 (s, 2H), 1.31 (s, 9H),

HR-FABMS Calcd. for C₁₇H₂₀N₃O (M⁺+H): 282.3602, Found: 282.1608.

### N¹-(5-methoxybenzo[d]oxazol-2-yl)benzene-1,4-diamine (3q)

Dark brown solid (51.3%),

mp 192-201° C.,

¹H-NMR (DMSO-d₆, 400 MHz) δ 9.99 (brs, 1H), 7.34 (dt, J=9.6, 2.6 Hz, 2H), 7.27 (d, J=8.4 Hz, 1H), 6.93 (d, J=2.4 Hz, 1H), 6.60 (d, J=2.8 Hz, 1H), 6.57 (dt, J=9.2, 2.6 Hz, 2H), 4.83 (s, 2H), 3.75 (s, 3H),

HR-FABMS Calcd. for C₁₄H₁₄N₃O₂ (M⁺+H): 256.2799.

### Example 6: Synthesis of 2-methoxy-N¹-(substituted-benzo[d]oxazol-2-yl)benzene-1,4-diamine Derivatives (3h-m)

A series of N¹-(substituted-benzo[d]oxazol-2-yl)benzene-1,4-diamine derivatives (3h-m) were obtained in a similar manner as in Example 5 above using each of the substituted-N-(2-methoxy-4-nitrophenyl)benzo[d]oxazol-2-amine derivatives (2h-m, 1 eq.) prepared according to Example 4 above.

### N¹-(benzo[d]oxazol-2-yl)-2-methoxybenzene-1,4-diamine (3h)

Grey solid (58.2%),

mp 110-112° C.

¹H-NMR (DMSO-d₆, 400 MHz) δ 9.06 (s, 1H), 7.33 (t, J=8.0 Hz, 2H), 7.25 (d, J=7.6 Hz, 1H), 7.14-7.10 (m, 1H), 7.02-6.97 (m, 1H), 6.31 (d, J=2.4 Hz, 1H), 6.17 (dd, J=8.8, 2.4 Hz, 1H), 5.05 (s, 2H), 3.70 (s, 3H),

HR-FABMS Calcd. for C₁₄H₁₄N₃O₂ (M⁺+H): 256.1081, Found: 256.1082.

### N¹-(5-fluorobenzo[d]oxazol-2-v)-2-methoxybenzene-1,4-diamine (3i)

White solid (41.5%),

mp 118-120° C.,

¹H-NMR (DMSO-d₆, 400 MHz) δ 9.25 (s, 1H), 7.34 (dd, J=8.8, 4.8 Hz, 1H), 7.246 (d, J=8.4 Hz, 1H), 7.07 (dd, J=5.6, 2.4 Hz, 1H), 6.82-6.76 (m, 1H), 6.31 (d, J=2.4 Hz, 1H), 6.17 (dd, J=8.4, 2.4 Hz, 1H), 5.09 (s, 2H), 3.70 (s, 3H),

HR-FABMS Calcd. for C₁₄H₁₃FN₃O₂ (M⁺+H): 274.0986, Found: 274.0989.

### N¹-(5-chlorobenzo[d]oxazol-2-v)-2-methoxybenzene-1,4-diamine (3j)

Grey solid (42.3%),

mp 117-120° C.,

¹H-NMR (DMSO-d₆, 400 MHz) δ 9.45 (s, 2H), 9.29 (s, 1H), 7.43 (d, J=8.0 Hz, 2H), 7.32 (d, J=2.0 Hz, 1H), 7.06-7.03 (m, 1H), 6.46 (s, 1H), 6.34 (d, J=8.0 Hz, 1H), 3.73 (s, 3H),

HR-FABMS Calcd. for C₁₄H₁₃ClN₃O₂ (M⁺+H): 290.0691, Found: 290.0692.

### 2-methoxy-N¹-(5-methylbenzo[d]oxazol-2-yl)benzene-1,4-diamine (3k)

Brown solid (44.4%),

mp 123-125° C.,

¹H-NMR (DMSO-d₆, 400 MHz) δ 8.98 (s, 1H), 7.31 (d, J=8.4 Hz, 1H), 7.20 (d, J=8.4 Hz, 1H), 7.05 (s, 1H), 6.80 (d, J=9.2 Hz, 1H), 6.31 (d, J=2.4 Hz, 1H), 6.17 (dd, J=8.4, 2.4 Hz, 1H), 5.05 (s, 2H), 3.70 (s, 3H), 2.32 (s, 3H),

HR-FABMS Calcd. for C₁₅H₁₆N₃O₂ (M⁺+H): 270.1237, Found: 270.1238.

### 2-methoxy-N¹-(6-methylbenzo[d]oxazol-2-yl)benzene-1,4-diamine (31)

Grey solid (57.0%),

mp 186-189° C.,

¹H-NMR (DMSO-d₆, 400 MHz) δ 8.94 (s, 1H), 7.34 (d, J=8.8 Hz, 1H), 7.12 (d, J=8.0 Hz, 1H), 6.93 (dd, J=7.6, 0.8 Hz, 1H), 6.31 (d, J=2.4 Hz, 1H), 6.17 (dd, J=8.0, 2.4 Hz, 1H), 5.03 (s, 2H), 3.70 (s, 3H), 2.34 (s, 3H),

HR-FABMS Calcd. for C₁₅H₁₆N₃O₂ (M⁺+H): 270.1237, Found: 270.1240.

### N¹-(5-tert-butylbenzo[d]oxazol-2-yl)-2-methoxybenzene-1,4-diamine (3m)

Brown solid (68.8%),

mp 185-188° C.,

¹H-NMR (DMSO-d₆, 400 MHz) δ 8.98 (s, 1H), 7.35 (d, J=8.0 Hz, 1H), 7.28 (d, J=1.6 Hz, 1H), 7.23 (d, J=8.4 Hz, 1H), 7.03 (dd, J=8.8, 1.6 Hz, 1H), 6.31 (d, J=2.4 Hz, 1H), 6.17 (dd, J=8.4, 2.4 Hz, 1H), 5.04 (s, 2H), 3.70 (s, 3H), 1.29 (s, 9H),

HR-FABMS Calcd. for C₁₈H₂₂N₃O₂ (M⁺+H): 312.1707, Found: 312.1707.

### Example 7: Synthesis of tert-butyl 4-(4-(substituted-benzo[d]oxazol-2-ylamino)phenylamino)-4-oxobutylcarbamate Derivatives (4a-g)

First, the N¹-(substituted benzo[d]oxazol-2-yl)benzene-1,4-diamine derivatives (3a-g, 100 mg, 1 eq.) prepared according to Example 5 above, Boc-GABA-OH (1 eq.) and PyBOP (1.2 eq.) were added to 10 mL of dimethylformamide and stirred at 0° C. Diisopropylethylamine (2 eq.) was added to the reaction mixture, and the reaction mixture was stirred at room temperature for 16 hours. After the reaction was completed, 10% HCl solution was added to the reaction mixture, extracted with ethyl acetate, and washed with sodium bicarbonate solution and saline. The resultant was dried over anhydrous MgSO₄ and filtered, then the organic solvent was removed under reduced pressure to obtain Compounds 4a-g.

### tert-butyl 4-(4-(benzo[d]oxazol-2-ylamino)phenylamino)-4-oxobutylcarbamate (4a)

Bright yellow solid (38.1%),

mp 124-126° C.,

¹H-NMR (DMSO-d₆, 400 MHz) δ 10.50 (s, 1H), 9.83 (s, 1H), 7.65 (d, J=4.4 Hz, 2H), 7.57 (d, J=9.2 Hz, 2H), 7.47-7.41 (m, 2H), 7.23-7.19 (m, 1H), 7.13-7.09 (m, 1H), 6.83 (s, 1H), 2.95 (t, J=6.4 Hz, 2H), 2.28 (t, J=7.6 Hz, 2H), 1.69 (t, J=7.2 Hz, 2H), 1.38 (s, 9H),

HR-FABMS Calcd. for C₂₂H₂₇N₄O₄ (M⁺+H): 411.2027, Found: 411.2025.

### tert-butyl 4-(4-(5-fluorobenzo[d]oxazol-2-ylamino)phenylamino)-4-oxobutylcarbamate (4b)

Green solid (75.7%),

mp 185-180° C.,

¹H-NMR (DMSO-d₆, 400 MHz) δ 10.62 (s, 1H), 9.84 (s, 1H), 7.63 (d, J=8.8 Hz, 2H), 7.58 (d, J=9.2 Hz, 2H), 7.47 (dd, J=8.8, 4.4 Hz, 1H), 7.27 (dd, J=9.2, 2.4 Hz, 1H), 6.94-6.89 (m, 1H), 6.83 (s, 1H), 2.96 (d, J=6.4 Hz, 2H), 2.28 (t, J=7.2 Hz, 2H), 1.69 (t, J=7.2 Hz, 2H), 1.38 (s, 9H),

HR-FABMS Calcd. for C₂₂H₂₆FN₄O₄ (M⁺+H): 429.1933, Found: 429.1931.

### tert-butyl 4-(4-(4-methylbenzo[d]oxazol-2-ylamino)phenylamino)-4-oxobutylcarbamate (4c)

Grey solid (33.9%),

mp 127-129° C.,

¹H-NMR (DMSO-d₆, 400 MHz) δ 10.43 (s, 1H), 9.83 (s, 1H), 7.67 (d, J=8.8 Hz, 2H), 7.57 (d, J=9.2 Hz, 2H), 7.27 (d, J=7.2 Hz, 1H), 7.04-6.98 (m, 2H), 6.83 (s, 1H), 2.95 (t, J=6.4 Hz, 2H), 2.46 (s, 3H), 2.28 (t, J=7.6 Hz, 2H), 1.75-1.67 (m, 2H), 1.38 (s, 9H),

HR-FABMS Calcd. for C₂₃H₂₉N₄O₄ (M⁺+H): 425.2183, Found: 425.2190.

### tert-butyl 4-(4-(5-methylbenzo[d]oxazol-2-ylamino)phenylamino)-4-oxobutylcarbamate (4d)

White powder (34.5%),

mp 186-189° C.,

¹H-NMR (DMSO-d₆, 400 MHz) δ 10.43 (s, 1H), 9.82 (s, 1H), 7.63 (d, J=9.2 Hz, 2H), 7.56 (d, J=9.2 Hz, 2H), 7.32 (d, J=8.4 Hz, 1H), 7.23 (s, 1H), 6.91 (d, J=8.4 Hz, 1H), 6.83 (s, 1H), 2.95 (t, J=7.2 Hz, 2H), 2.36 (s, 3H), 2.27 (t, J=7.6 Hz, 2H), 1.69 (t, J=7.2 Hz, 2H), 1.38 (s, 9H),

HR-FABMS Calcd. for C₂₃H₂₉N₄O₄ (M⁺+H): 425.2183, Found: 425.2184.

### tert-butyl 4-(4-(6-methylbenzo[d]oxazol-2-ylamino)phenylamino)-4-oxobutylcarbamate (4e)

Bright brown solid (50.9%),

mp 173-176° C.,

¹H-NMR (DMSO-d₆, 400 MHz) δ 10.39 (s, 1H), 9.79 (s, 1H), 7.65-7.60 (m, 2H), 7.55-7.52 (m, 2H), 7.26 (t, J=7.6 Hz, 2H), 6.99-6.96 (m, 1H), 6.80 (s, 1H), 2.92 (t, J=5.6 Hz, 2H), 2.35 (s, 3H), 2.24 (t, J=7.6 Hz, 2H), 1.65 (d, J=7.2 Hz, 2H), 1.35 (s, 9H),

HR-FABMS Calcd. for C₂₃H₂₉N₄O₄ (M⁺+H): 425.2183, Found: 425.2184.

### tert-butyl 4-(4-(5-tert-butylbenzo[d]oxazol-2-ylamino)phenylamino)-4-oxobutylcarbamate (4f)

Brown oil (60.2%),

¹H-NMR (DMSO-d₆, 400 MHz) δ 10.43 (s, 1H), 9.82 (s, 1H), 7.65 (d, J=9.2 Hz, 2H), 7.56 (d, J=8.8 Hz, 2H), 7.45 (s, 1H), 7.4 (d, J=8.4 Hz, 1H), 7.14 (d, J=6.8 Hz, 1H), 6.83 (s, 1H), 2.96 (d, J=6.4 Hz, 2H), 2.27 (t, J=6.8 Hz, 2H), 1.68 (d, J=7.2 Hz, 2H), 1.38 (s, 9H), 1.32 (s, 9H),

HR-FABMS Calcd. for C₂₆H₃₅N₄O₄ (M⁺+H): 467.2653, Found: 467.2656.

### tert-butyl 4-(4-(5-methoxybenzo[d]oxazol-2-ylamino)phenylamino)-4-oxobutylcarbamate (4g)

Ivory solid (90.0%),

mp 190-192° C.,

¹H-NMR (DMSO-d₆, 400 MHz) δ 10.45 (s, 1H), 9.82 (s, 1H), 7.63 (d, J=8.8 Hz, 2H), 7.56 (d, J=9.2 Hz, 2H), 7.34 (d, J=9.2 Hz, 1H), 7.02 (d, J=2.4 Hz, 1H), 6.83 (t, J=4.8 Hz, 1H), 6.66 (dd, J=8.4, 2.8 Hz, 1H), 3.77 (s, 3H), 2.96 (dd, J=13.2, 6.8 Hz, 2H), 2.27 (t, J=7.6 Hz, 2H), 1.69 (t, J=7.2 Hz, 2H), 1.38 (s, 9H),

HR-FABMS Calcd. for C₂₃H₂₉N₄O₅ (M⁺+H): 441.2132, Found: 441.2128.

### Example 8: Synthesis of tert-butyl 4-(4-(substituted-benzo[d]oxazol-2-ylamino)-3-methoxyphenylamino)-4-oxobutylcarbamate Derivatives (4h-m)

A series of tert-butyl 4-(4-(substituted-benzo[d]oxazol-2-ylamino)phenylamino)-4-oxobutylcarbamate derivatives (4h-m) were obtained in a similar manner as in Example 7 above using each of the 2-methoxy-N¹-(substituted-benzo[d]oxazol-2-yl)benzene-1,4-diamine derivatives (3h-m, 1 eq.) obtained according to Example 6 above.

### tert-butyl 4-(4-(benzo[d]oxazol-2-ylamino)-3-methoxyphenylamino)-4-oxobutylcarbamate (4h)

Brown oil (81.1%),

mp 110-112° C.,

¹H-NMR (DMSO-d₆, 400 MHz) δ 9.90 (s, 1H), 9.50 (s, 1H), 7.93 (d, J=8.4 Hz, 1H), 7.51 (d, J=2.0 Hz, 1H), 7.43-7.35 (m, 1H), 7.19 (dd, J=7.6, 0.8 Hz, 1H), 7.13 (dd, J=8.8, 2.0 Hz, 1H), 7.07 (dd, J=8.0, 1.2 Hz, 1H), 6.84 (s, 1H), 3.80 (s, 3H), 2.97 (d, J=6.4 Hz, 2H), 2.29 (s, 2H), 1.69 (s, 2H), 1.38 (s, 9H),

HR-FABMS Calcd. for C₂₃H₂₉N₄O₅ (M⁺+H): 441.2132, Found: 441.2137.

### tert-butyl 4-(4-(5-fluorobenzo[d]oxazol-2-ylamino)-3-methoxyphenylamino)-4-oxobutyl)-carbamate (4i)

Bright brown solid (59.5%),

mp 166-170° C.,

¹H-NMR (DMSO-d₆, 400 MHz) δ 9.91 (s, 1H), 9.67 (s, 1H), 7.84 (d, J=8.4 Hz, 1H), 7.51 (d, J=2.0 Hz, 1H), 7.42 (dd, J=8.8, 4.4 Hz, 1H), 7.19 (dd, J=9.2, 2.8 Hz, 1H), 7.13 (dd, J=8.8, 2.0 Hz, 1H), 6.90-6.84 (m, 2H), 3.80 (s, 3H), 2.96 (t, J=6.8 Hz, 2H), 2.29 (t, J=7.6 Hz, 2H), 1.68 (d, J=6.8 Hz, 2H), 1.38 (s, 9H),

HR-FABMS Calcd. for C₂₄H₃₁N₄O₅ (M⁺+H): 455.2289, Found: 455.2292.

### tert-butyl 4-(4-(5-chlorobenzo[d]oxazol-2-ylamino)-3-methoxyphenylamino)-4-oxobutyl)-carbamate (4i)

Purple solid (85.4%),

mp 60-63° C.,

¹H-NMR (DMSO-d₆, 400 MHz) δ 9.92 (s, 1H), 9.73 (s, 1H), 7.94-7.81 (m, 1H), 7.51 (d, J=2.4 Hz, 1H), 7.45-7.40 (m, 1H), 7.20-7.08 (m, 2H), 6.84 (s, 1H), 3.79 (s, 3H), 2.96 (t, J=6.0 Hz, 2H), 2.29 (t, J=7.6 Hz, 2H), 1.68 (d, J=7.2 Hz, 2H), 1.38 (s, 9H),

HR-FABMS Calcd. for C₂₃H₂₈ClN₄O₅ (M⁺+H): 475.1743, Found: 475.1743.

### tert-butyl 4-(3-methoxy-4-(5-methylbenzo[d]oxazol-2-ylamino)phenylamino)-4-oxobutyl)-carbamate (4k)

White solid (73.9%),

mp 88-85° C.,

¹H-NMR (DMSO-d₆, 400 MHz) δ 9.90 (s, 1H), 9.43 (s, 1H), 7.92 (d, J=8.8 Hz, 1H), 7.50 (d, J=2.0 Hz, 1H), 7.28 (d, J=7.6 Hz, 1H), 7.12 (dd, J=8.8, 2.0 Hz, 1H), 6.83 (d, J=5.6 Hz, 1H), 3.80 (s, 3H), 3.31-2.99 (m, 2H), 2.35 (s, 3H), 2.29 (t, J=7.6 Hz, 2H), 1.69 (t, J=6.8 Hz, 2H), 1.38 (s, 9H),

HR-FABMS Calcd. for C₂₄H₃₁N₄O₅ (M⁺+H): 455.2289, Found: 455.2289.

### tert-butyl 4-(3-methoxy-4-(6-methylbenzo[d]oxazol-2-ylamino)phenylamino)-4-oxobutyl)-carbamate (41)

White solid (91.3%),

mp 150-153° C.,

¹H-NMR (DMSO-d₆, 400 MHz) δ 9.89 (s, 1H), 9.39 (s, 1H), 7.95 (d, J=8.4 Hz, 1H), 7.50 (d, J=2.0 Hz, 1H), 7.24 (d, J=8.4 Hz, 2H), 7.12 (dd, J=8.8, 2.4 Hz, 1H), 6.99 (d, J=6.8 Hz, 1H), 6.84 (s, 1H), 3.80 (s, 3H), 2.96 (t, J=6.4 Hz, 2H), 2.37 (s, 3H), 2.29 (t, J=7.6 Hz, 2H), 1.69 (t, J=7.6 Hz, 2H), 1.38 (s, 9H),

HR-FABMS Calcd. for C₂₄H₃₁N₄O₅ (M⁺+H): 455.2289, Found: 455.2292.

### tert-butyl 4-(4-(5-tert-butylbenzo[d]oxazol-2-ylamino)-3-methoxyphenylamino)-4-oxobutylcarbamate (4m)

White solid (83.7%),

mp 168-171° C.,

¹H-NMR (DMSO-d₆, 400 MHz) δ 9.89 (s, 1H), 9.43 (s, 1H), 7.96 (d, J=8.4 Hz, 1H), 7.49 (d, J=2.4 Hz, 1H), 7.39 (d, J=1.6 Hz, 1H), 7.31 (d, J=4.4 Hz, 1H), 7.14-7.09 (m, 2H), 6.84 (s, 1H), 3.80 (s, 3H), 2.99-2.94 (m, 2H), 2.29 (t, J=7.2 Hz, 2H), 1.69 (t, J=7.6 Hz, 2H), 1.382 (s, 9H),

HR-FABMS Calcd. for C₂₇H₃₇N₄O₅ (M⁺+H): 497.2758, Found: 497.2762.

### Example 9: Synthesis of 4-amino-N-(4-(substituted-benzo[d]oxazol-2-ylamino)phenyl)butanamide Derivatives (5a-g)

First, 4 M HCl-dioxane solution (3 mL) was added to the chloroform solution of each of the tert-butyl 4-(4-(substituted-benzo[d]oxazol-2-ylamino)phenylamino)-4-oxobutylcarbamate derivatives (4a-g, 100 mg, 1 eq.) prepared according to Example 7 above. The reaction mixture was stirred at room temperature for 3 hours. After the reaction was completed, the organic solvent was removed under reduced pressure to obtain Compounds 5a-g. 4-amino-N-(4-(benzo[d]oxazol-2-ylamino)phenyl)butanamide (5a)

Bright yellow solid (90.5%),

mp 110-112° C.,

¹H-NMR (DMSO-d₆, 400 MHz) δ 10.54 (s, 1H), 9.99 (s, 1H), 7.80 (s, 2H), 7.67 (d, J=8.8 Hz, 2H), 7.58 (d, J=8.8 Hz, 2H), 7.47 (d, J=7.6 Hz, 1H), 7.42 (d, J=7.2 Hz, 1H), 7.23-7.19 (m, 1H), 7.13-7.09 (m, 1H), 2.87-2.82 (m, 2H), 2.42 (t, J=7.2 Hz, 2H), 1.86 (t, J=7.6 Hz, 2H),

HR-FABMS Calcd. for C₁₇H₁₉N₄O₂ (M⁺+H): 311.1503, Found: 311.1505.

### 4-amino-N-(4-(5-fluorobenzo[d]oxazol-2-ylamino)phenyl)butanamide (5b)

Green solid (69.9%),

mp 115-118° C.,

¹H-NMR (DMSO-d₆, 400 MHz) δ 10.66 (s, 1H), 9.98 (s, 1H), 7.75 (s, 2H), 7.66-7.64 (m, 2H), 7.59-7.57 (m, 2H), 7.51-7.46 (m, 1H), 7.27 (dd, J=9.2, 2.4 Hz, 1H), 6.95-6.90 (m, 1H), 2.85 (dd, J=14.8, 6.0 Hz, 2H), 2.41 (t, J=7.2 Hz, 2H), 1.85 (t, J=7.6 Hz, 2H),

HR-FABMS Calcd. for C₁₇H₁₈FN₄O₂ (M⁺+H): 329.1408, Found: 329.1413.

### 4-amino-N-(4-(4-methylbenzo[d]oxazol-2-ylamino)phenyl)butanamide (5c)

Brown solid (34.8%),

mp 155-158° C.,

¹H-NMR (DMSO-d₆, 400 MHz) δ 10.47 (s, 1H), 9.98 (s, 1H), 7.79 (s, 2H), 7.68 (d, J=9.2 Hz, 2H), 7.58 (d, J=8.8 Hz, 2H), 7.27 (d, J=7.2 Hz, 1H), 7.04-6.98 (m, 2H), 2.83 (d, J=7.2 Hz, 2H), 2.46-2.40 (m, 2H), 1.88-1.83 (m, 2H),

HR-FABMS Calcd. for C₁₈H₂₁N₄O₂ (M⁺+H): 325.1659, Found: 325.1659.

### 4-amino-N-(4-(5-methylbenzo[d]oxazol-2-ylamino)phenyl)butanamide (5d)

Grey solid (56.5%),

mp 158-160° C.,

¹H-NMR (DMSO-d₆, 400 MHz) δ 10.48 (s, 1H), 10.00 (s, 1H), 7.83 (s, 2H), 7.65 (d, J=9.2 Hz, 2H), 7.58 (d, J=9.2 Hz, 2H), 7.33 (d, J=8.0 Hz, 1H), 6.91 (dd, J=8.0, 0.8 Hz, 1H), 2.85 (dd, J=14.8, 6.0 Hz, 2H), 2.42 (t, J=7.2 Hz, 2H), 2.37 (s, 3H), 1.86 (t, J=8.0 Hz, 2H),

HR-FABMS Calcd. for C₁₈H₂₁N₄O₂ (M⁺+H): 325.1659, Found: 325.1658.

### 4-amino-N-(4-(6-methylbenzo[d]oxazol-2-ylamino)phenyl)butanamide (5e)

Bright brown solid (43.5%),

mp 126-129° C.,

¹H-NMR (DMSO-d₆, 400 MHz) δ 10.46 (s, 1H), 9.99 (s, 1H), 7.82 (s, 2H), 7.67-7.64 (m, 2H), 7.58 (d, J=7.2 Hz, 2H), 7.29 (d, J=8.0 Hz, 2H), 7.03-7.01 (m, 1H), 2.85 (dd, J=14.8, 6.0 Hz, 2H), 2.43-2.38 (m, 5H), 1.86 (t, J=7.6 Hz, 2H),

HR-FABMS Calcd. for C₁₈H₂₁N₄O₂ (M⁺+H): 325.1659, Found: 325.1661.

### 4-amino-N-(4-(5-tert-butylbenzo[d]oxazol-2-ylamino)phenyl)butanamide (5f)

Brown solid (24.4%),

mp 165-168° C.,

¹H-NMR (DMSO-d₆, 400 MHz) δ 10.48 (s, 1H), 10.01 (s, 1H), 7.86 (s, 2H), 7.67 (d, J=8.8 Hz, 2H), 7.58 (d, J=9.2 Hz, 2H), 7.44 (d, J=1.6 Hz, 1H), 7.35 (d, J=8.4 Hz, 1H), 7.14 (dd, J=8.0, 1.6 Hz, 1H), 2.83 (s, 2H), 2.42 (s, 2H), 1.88-1.81 (m, 2H), 1.32 (s, 9H),

HR-FABMS Calcd. for C₂₁H₂₇N₄₀₂ (M⁺+H): 367.2129, Found: 367.2133.

### 4-amino-N-(4-(5-methoxybenzo[d]oxazol-2-ylamino)phenyl)butanamide (5q)

Ivory solid (84.7%),

mp 216-219′C,

¹H-NMR (DMSO-d₆, 400 MHz) δ 10.51 (s, 1H), 10.01 (s, 1H), 7.86 (s, 2H), 7.65 (d, J=9.2 Hz, 2H), 7.58 (d, J=8.8 Hz, 2H), 7.35 (d, =8.8 Hz, 1H), 7.01 (d, J=2.0 Hz, 1H), 6.67 (dd, J=8.4, 2.8 Hz, 1H), 3.77 (s, 3H), 2.87-2.80 (m, 2H), 2.42 (t, J=7.2 Hz, 2H), 1.86 (t, J=8.0 Hz, 2H),

HR-FABMS Calcd. for C₁₈H₂₁N₄O₃ (M⁺+H): 341.1608, Found: 325.1607.

### Example 10: Synthesis of 4-amino-N-(4-(substituted-benzo[d]oxazol-2-ylamino)-3-methoxyphenyl)butanamide Derivatives (5h-m)

A series of 4-amino-N-(4-(substituted-benzo[d]oxazol-2-ylamino)phenyl)butanamide derivatives (5h-m) were obtained in a similar manner as in Example 9 above using each of the tert-butyl 4-(4-(substituted-benzo[d]oxazol-2-ylamino)-3-methoxyphenylamino)-4-oxobutylcarbamate derivatives (4h-m, 1 eq.) obtained according to Example 8 above.

### 4-amino-N-(4-(benzo[d]oxazol-2-ylamino)-3-methoxyphenyl)butanamide (5h)

White solid (87.7%),

mp 98-100° C.,

¹H-NMR (DMSO-d₆, 400 MHz) δ 10.08 (s, 1H), 9.57 (s, 1H), 7.95 (d, J=8.4 Hz, 1H), 7.83 (s, 2H), 7.50 (d, J=2.4 Hz, 1H), 7.43 (d, J=7.2 Hz, 1H), 7.36 (d, J=7.6 Hz, 1H), 7.21-7.15 (m, 1H), 7.07-7.02 (m, 1H), 3.81 (s, 3H), 2.89-2.83 (m, 2H), 2.44 (t, J=14.4 Hz, 2H), 1.87 (t, J=8.0 Hz, 2H),

HR-FABMS Calcd. for C₁₈H₂₁N₄O₃ (M⁺+H): 341.1608, Found: 341.1609.

### 4-amino-N-(4-(5-fluorobenzo[d]oxazol-2-ylamino)-3-methoxyphenyl)butanamide (5i)

White solid (56.4%),

mp 193-195° C.,

¹H-NMR (DMSO-d₆, 400 MHz) δ 10.10 (s, 1H), 9.70 (s, 1H), 7.86 (d, J=8.4 Hz, 3H), 7.50 (d, J=2.4 Hz, 1H), 7.42 (dd, J=8.0, 3.6 Hz, 1H), 7.21-7.15 (m, 2H), 6.91-6.85 (m, 1H), 3.80 (s, 3H), 2.85 (d, J=8.8 Hz, 2H), 2.44 (t, J=7.2 Hz, 2H), 1.87 (t, J=8.0 Hz, 2H),

HR-FABMS Calcd. for C₂₃H₂₈FN₄O₅ (M⁺+H): 459.2038, Found: 459.2041.

### 4-amino-N-(4-(5-chlorobenzo[d]oxazol-2-ylamino)-3-methoxyphenyl)butanamide (5j)

Purple solid (37.3%),

mp 110-113° C.,

¹H-NMR (DMSO-d₆, 400 MHz) δ 10.09 (s, 1H), 9.75 (s, 1H), 7.83 (s, 2H), 7.50 (s, 1H), 7.45 (d, J=8.4 Hz, 1H), 7.19-7.15 (m, 1H), 7.07 (dd, J=8.4, 2.0 Hz, 1H), 3.80 (s, 3H), 2.88-2.82 (m, 2H), 2.44 (t, J=7.6 Hz, 2H), 1.87 (t, J=7.6 Hz, 2H),

HR-FABMS Calcd. for C₁₈H₂₀ClN₄O₃ (M⁺+H): 375.1218, Found: 375.1218.

### 4-amino-N-(3-methoxy-4-(5-methylbenzo[d]oxazol-2-ylamino)phenyl)butanamide (5k)

White solid (95.7%),

mp 166-168° C.,

¹H-NMR (DMSO-d₆, 400 MHz) δ 10.05 (s, 1H), 9.50 (s, 1H), 7.93 (d, J=8.8 Hz, 1H), 7.79 (s, 2H), 7.49 (d, J=2.4 Hz, 1H), 7.29 (d, J=8.4 Hz, 1H), 7.16 (dd, J=8.4, 2.4 Hz, 2H), 6.89 (dd, J=8.0, 0.8 Hz, 1H), 3.80 (s, 3H), 2.88-2.83 (m, 3H), 2.43 (t, J=7.2 Hz, 2H), 2.35 (s, 3H), 1.87 (t, J=8.0 Hz, 2H),

HR-FABMS Calcd. for C₁₉H₂₃N₄O₃ (M⁺+H): 355.1765, Found: 355.1762.

### 4-amino-N-(3-methoxy-4-(6-methylbenzo[d]oxazol-2-ylamino)phenyl)butanamide (51)

White solid (69.2%),

mp 215-217′C,

¹H-NMR (DMSO-d₆, 400 MHz) δ 10.20 (s, 1H), 9.78 (s, 1H), 8.01 (s, 2H), 7.89 (d, J=8.8 Hz, 1H), 7.53 (d, J=2.0 Hz, 1H), 7.29 (s, 1H), 7.24 (d, J=7.6 Hz, 1H), 7.19 (dd, J=9.2, 2.0 Hz, 1H), 7.02 (d, J=7.2 Hz, 1H), 3.80 (s, 3H), 2.83 (t, J=7.2 Hz, 2H), 2.45 (t, J=7.2 Hz, 2H), 2.37 (s, 3H), 1.88 (t, J=7.2 Hz, 2H),

HR-FABMS Calcd. for C₁₉H₂₃N₄O₃ (M⁺+H): 355.1765, Found: 355.1765.

### 4-amino-N-(4-(5-tert-butylbenzo[d]oxazol-2-ylamino)phenyl)butanamide (5m)

Brown solid (24.4%),

mp 165-168° C.,

¹H-NMR (DMSO-d₆, 400 MHz) δ 10.48 (s, 1H), 10.01 (s, 1H), 7.86 (s, 2H), 7.67 (d, J=8.8 Hz, 2H), 7.58 (d, J=9.2 Hz, 2H), 7.44 (d, J=1.6 Hz, 1H), 7.35 (d, J=8.4 Hz, 1H), 7.14 (dd, J=8.0, 1.6 Hz, 1H), 2.83 (s, 2H), 2.42 (s, 2H), 1.88-1.81 (m, 2H), 1.32 (s, 9H),

HR-FABMS Calcd. for C₂₁H₂₇N₄O₂ (M⁺+H): 367.2129, Found: 367.2133.

### Experimental Example 1: Cell Cultivation

Human keratinocytes HaCaT or alpha mouse liver 12 (AML-12) cells were obtained from Cell Lines Service GmbH (Eppelheim, Germany). The cells were cultured in Dulbecco's modified Eagle's medium (DMEM, HyClone) supplemented with 10% fetal bovine serum (HyClone) (HaCaT cells) and 1% penicillin/streptomycin (HyClone) and maintained at 37° C. in a humidified atmosphere containing 5% CO₂. AML-12 cells were pretreated with compounds at a concentration of 10 μM 1 hour before LPS treatment and then treated with LPS (100 ng/mL) for 24 hours.

### Experimental Example 2: Assessment of Cell Viability by MTT Assay

Cell viability was measured by MTT assay. HaCaT cells were seeded in 96-well plates at a density of 5.0×10⁴ cells/well. The cells were treated with the indicated concentrations of compounds 1 hour before LPS treatment on the following day. LPS (Sigma-Aldrich) was left in the growth medium for 6 hours, and then the cells were gently washed twice with growth medium and incubated with 0.5 mg/mL MTT (Sigma-Aldrich) at 37° C. for 1 hour. Formazan crystals formed by active mitochondria were dissolved in DMSO and absorbance at A540 was measured for each well using a spectrophotometer.

### Experimental Example 3: Animal Model

10-week-old male C₅₇BL/6J mice (Jackson Laboratory, Bar Harbor, ME, USA) were used in the experiment. All animal experiments were performed in accordance with the protocol approved by the Institutional Animal Care and Use Committee of Ewha Womans University (EWHA IACUC 19-001). The mice were housed in a temperature-controlled room (20 to 22° C. with a 12 h light:12 h dark cycle). The compounds prepared according to Examples 1 to 10 were diluted with olive oil (vehicle, Sigma) to a volume of 200 μL (10 mg/kg) and administered to mice (male C57BL/6J, 8 weeks old) 1 hour before LPS administration. LPS (1 mg/kg, 6 h) was resuspended in saline and injected intraperitoneally at a dose of 1 mg/kg of body weight. At the end of the experiment, the mice were sacrificed (tribromoethanol, 250 mg/kg, i.p.) and the liver and blood were collected for further analysis.

### Experimental Example 4: Measurement of Blood Parameters

Blood was collected from the inferior vena cava and centrifuged at 3000 rpm for 15 minutes at 4° C. to separate plasma. The concentrations of plasma alanine aminotransferase (ALT) and aspartate transaminase (AST) were determined using an EnzyChrom™ assay kit (BioAssay Systems, Hayward, CA, USA) according to the manufacturer's recommendations.

### Experimental Example 5: Histology and Immunohistology

First, 4% paraformaldehyde-fixed liver tissues were embedded in paraffin according to standard procedures. 4-micron-thick liver tissue sections were prepared, and immunohistochemical staining was performed according to the manufacturer's instructions. 4-micron-thick liver tissue sections were stained with hematoxylin and eosin (H&E) or subjected to immunohistochemical characterization using an antibody specific for F4/80 (Abcam, Cambridge, MA, UK). All steps were performed at room temperature, and the tissues were washed with tap water after each step. The sections were photographed using a Zeiss microscope (Carl Ziess, Thronwood, NY, USA) equipped with AxioCam software.

### Experimental Example 6: Immunoblotting

Protein levels in cells and mouse tissues were determined by immunoblot analysis. The cells and tissues were lysed with cold lysis buffer (20 mM HEPES pH 7.0, 0.15 M NaCl, 10% glycerol, 1% Triton X-100, 1 mM EDTA, 1 mM EGTA, 10 mM β-phosphoglycerate, 1 mM Na₃VO₄, 5 mM NaF, 1 μg/mL aprotinin, 1 μg/mL leupeptin, 100 μM PMSF) using a Polytron homogenizer or a sonicator. The homogenates were centrifuged at 15,000 rpm at 4° C. for 15 min. After quantifying the protein concentrations in the lysates (supernatant) using the Bradford assay (Bio-Rad, CA, USA), the lysates were mixed with a sample buffer (62.5 mM Tris-HCl pH 6.8, 10% glycerol, 2% sodium dodecyl sulfate, 0.0125% bromophenol blue, 2.5% β-mercaptoethanol) and heated at 95° C. for 5 minutes. The samples were loaded onto sodium dodecyl sulfate-polyacrylamide gel electrophoresis (SDS-PAGE) gel and separated by electrophoresis in an SDS buffer (3 g/L Tris, 14.35 g/L glycine, 1 g/L SDS). The proteins were transferred onto an activated polyvinylidene difluoride (PVDF) membrane with a pore size of 0.45 μm (Millipore, Darmstadt, Germany) using a transfer buffer containing methanol (3.03 g/L Tris, 14.17 g/L glycine, 20% methanol). The membrane was incubated in Tween-20 Tris-buffered saline (TTBS) containing 5% bovine serum albumin (BSA) for 20 minutes at room temperature using a rocker, and then incubated with antibodies (1:2000 dilution) on a rocker overnight at 4° C. Immune complexes were detected with horseradish peroxidase (HRP)-conjugated secondary antibodies (Bio-Rad, Hercules, CA, USA) and enhanced with chemiluminescence reagents (Ab Frontier, Daejeon, Korea) using IQ800 (GE Healthcare, Sweden). The abundance of the target protein was quantified by densitometric analysis of the immunoblot. Bradford assay (SpectraMax M2 Microplate Reader, Molecular Devices) data were obtained from the Fluorescence Core Imaging Center at Ewha Womans University.

### Experimental Example 7: Quantitative Real-Time PCR Analysis

Total RNA was isolated from tissues using TRIzol reagent (Invitrogen), and reverse transcription (RT) and real-time PCR analysis were performed using the ABI PRISM 7700 system (PE Biosystems). Data were normalized to the amount of GAPDH mRNA. The primers used are shown in Table 1 below.

TABLE 1

| Gene | Forward Primer | Reverse Primer |
|---|---|---|
| GAPDH | AGAACATCATCC | GGTCCTCAGTGTA |
| | CTGCATCC | GCCCAAG |
| | (SEQ ID NO: 1) | (SEQ ID NO: 2) |
| IL-1β | TCGTGCTGTCGG | GTCGTTGCTTGGT |
| | ACCCATAT | TCTCCTTGT |
| | (SEQ ID NO: 3) | (SEQ ID NO: 4) |
| IL-6 | ACAACCACGGCCT | CACGATTTCCCAG |
| | TCCCTACTT | AGAACATGTG |
| | (SEQ ID NO: 5) | (SEQ ID NO: 6) |
| TNF-α | GCCACCACGCTCT | GGTGTGGGTGAG |
| | TCTG | GAGCA |
| | (SEQ ID NO: 7) | (SEQ ID NO: 8) |

### Experimental Example 8: Statistical Analysis

All quantitative data were analyzed using the GraphPad Prism software and expressed as mean±standard deviation (SD). Statistical significance of data between control and treatment groups was determined using one-way analysis of variance (ANOVA), and then the differences between the experimental groups were determined using the Tukey posthoc test. A p-value of less than 0.05 was considered statistically significant.

### Example 11: Inhibition of IL-6 and IL-1β mRNA Expression Levels in Vitro

13 novel synthetic compounds (Compounds 5a to 5m) and 3 synthetic intermediates (Compounds 4d, 4e, and 41) were screened by measuring the mRNA expression levels of IL-6 and IL-1β. Human keratinocyte HaCaT cell lines were used to evaluate the mRNA expression inhibition effect. Compounds 5d, 5c, 5f, 5m, and synthetic intermediate 4d showed strong activity (Table 2). Each compound was administered at a concentration of 10 μM. The mRNA expression levels of IL-6 when LPS was administered after pretreatment with each compound were 2.4% for Compound 4d, 2.08% for Compound 5c, 3.39% for Compound 5d, 3.26% for Compound 5f, and 4.07% for Compound 5m, which were significantly lower than 100%, when inflammation was induced by LPS administration alone and the increased mRNA expression level of IL-6 was set to 100%.

TABLE 2

mRNA Expression (%) (10 μM)

| | | |
|---|---|---|
| Compound | IL-6 | IL-1β |

| | |
|---|---|
| LPS | 100.0 |

| | | |
|---|---|---|
| 4d | 2.40 | 0.03 |
| 5c | 2.08 | 0.79 |
| 5d | 3.39 | 0.03 |
| 5f | 3.26 | 2.83 |
| 5m | 4.07 | 0.49 |

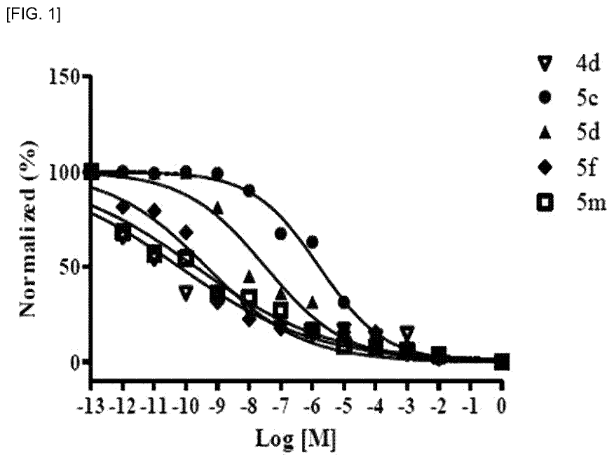

Dose-response studies were performed for the above five compounds at different concentrations to obtain IC₅₀ values (μM) (FIG. 1 and Table 3). The IC₅₀ of Compound 4d was 6.04×10⁻⁵, the IC₅₀ of Compound 5c was 1.64, the IC₅₀ of Compound 5d was 3.27×10⁻², the IC₅₀ of Compound 5f was 4.44×10⁻⁴, and the IC₅₀ of Compound 5m was 1.99×10⁻⁴. Cell viability was measured by MTT assay to confirm that the compounds did not exhibit toxicity.

TABLE 3

| Compound | Log [M] | IC₅₀ (μM) |
|---|---|---|
| 4d | −10.22 | 6.04 × 10⁻⁵ |
| 5c | −5.78 | 1.64 |
| 5d | −7.48 | 3.27 × 10⁻² |
| 5f | −9.35 | 4.44 × 10⁻⁴ |
| 5m | −9.70 | 1.99 × 10⁻⁴ |

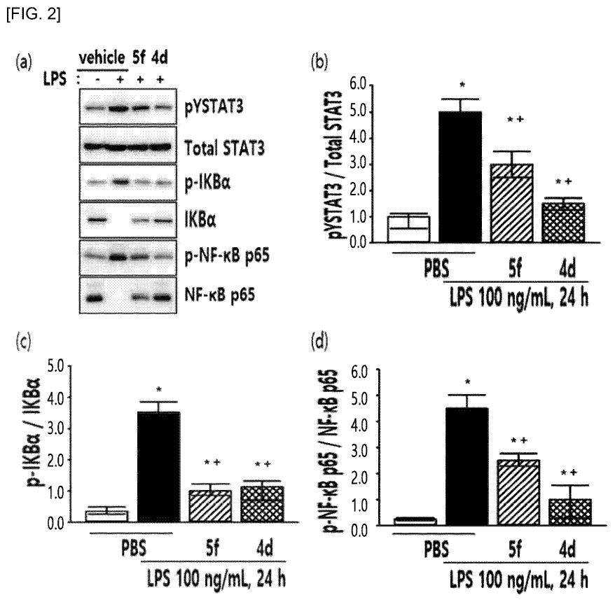

In order to confirm the inhibitory effect of two compounds (Compounds 4d and 5f) among the five compounds above on LPS-induced inflammation signaling in human liver hepatocytes AML-12 cells, the inhibitory effect of these compounds on hepatocytes was tested using a concentration range where no cytotoxicity was observed. Western blotting assay was performed to confirm the change in STAT3 or NE-KB activity, and it was confirmed that the phosphorylation of STAT3 or IκB-α was prevented in AML-12 cells treated with the two compounds, and statistical significance was confirmed in the groups treated with 10 μM of the compounds. As shown in FIG. 2, the expression levels of pY-STAT3, p-IκBα, and p-NF-κB p65 in AML-12 cells were significantly down-regulated by Compounds 5f and 4d. Many previous studies have shown that LPS induces inflammatory responses by activating the STAT3 or NF-κB pathway, and from the result, it could be inferred that Compounds 4d and 5f of the present disclosure regulate inflammation by inhibiting STAT3/NF-κB activity.

### Example 12: Inhibition of IL-6 and IL-1β mRNA Expression Levels in Mice in Vivo

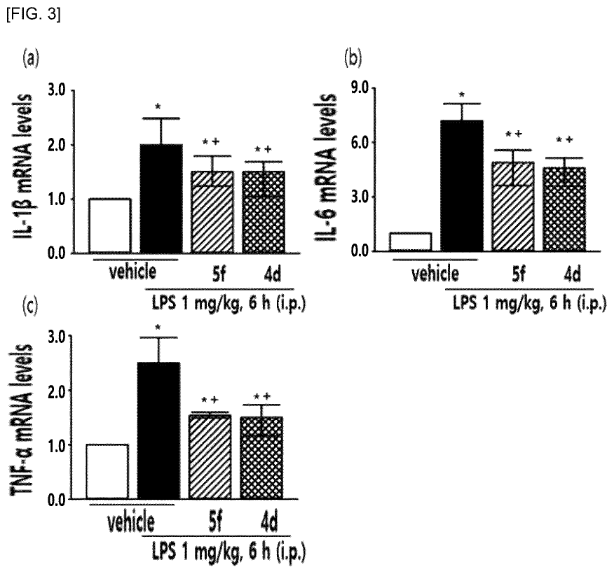

2 compounds (Compounds 4d and 5f) were selected for in vivo assay. These compounds were diluted in olive oil and administered to mice 1 hour before the administration of LPS (1 mg/kg, 6 h). LPS was resuspended in saline and injected intraperitoneally (i.p.) at a dose of 1 mg/kg of body weight. At the end of the experiment, the mice were sacrificed and blood and liver were collected. In order to evaluate the anti-inflammatory activity in mice, the mRNA expressions of IL-1β, IL-6, and TNF-α were measured, and the results are shown in FIG. 3.

### Example 13: Liver Morphology and Macrophage Infiltration

Liver histology showed inflammatory activity compared to the control group by LPS treatment (FIG. 3a). As a result of LPS administration, macrophage infiltration into the liver tissue was observed. When Compounds 5f and 4d were administered, the inflammatory response was improved compared to when LPS was administered alone.

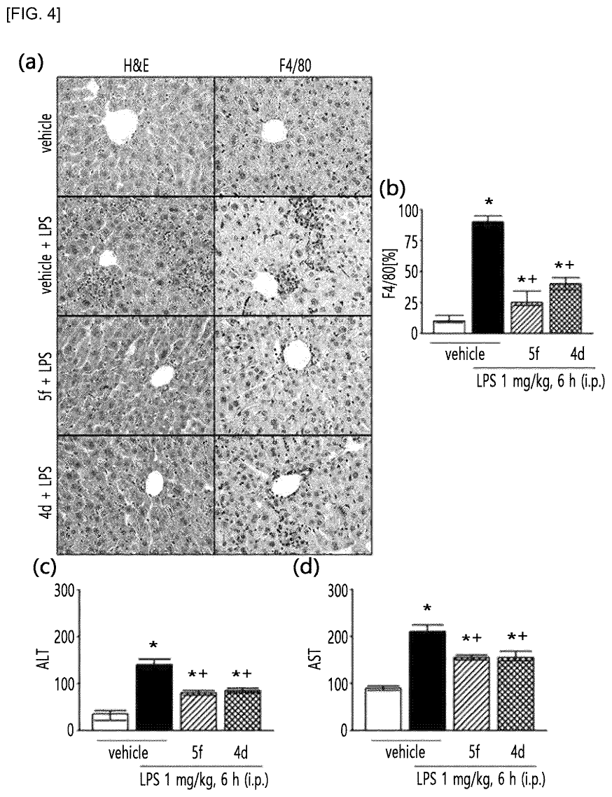

In order to characterize the phenotype of macrophages accumulated in LPS-induced inflammation, immunohistochemical studies were performed using antibodies against F4/80 as a marker of mature macrophages (FIG. 4b). When LPS was administered alone to induce an inflammatory response, hepatic macrophage accumulation increased. As a result, there was a marked elevation of the number of F4/80-positive cells. When Compounds 5f and 4d were administered, a significant decrease in F4/80-positive cells was observed.

Consistent with the altered histological characteristics, plasma alanine aminotransferase (ALT) and aspartate transaminase (AST) levels increased after LPS administration (FIGS. 4c and 4d), and when Compounds 5f and 4d were administered, ALT and AST levels decreased.

### Conclusion

In the present disclosure, 13 novel compounds including a 4-amino-N-(4-(benzo[d]oxazol-2-ylamino)phenyl)butanamide moiety were synthesized, and their biological activities were evaluated. Among them, five compounds (Compounds 5d, 5c, 5f, 5m, and synthetic intermediate 4d) exhibited strong inhibitory activities on IL-13 and IL-6 mRNA expression in vitro. Furthermore, two compounds (Compounds 5f and 4d) significantly reduced the mRNA levels of IL-1β, IL-6, and TNF-α without hepatotoxicity in vivo. From these in vivo and in vitro test results, it was confirmed that these synthesized compounds were effective in suppressing representative inflammatory cytokines.

### DISCUSSION

In the present disclosure, novel compounds having a benzoxazole moiety were synthesized, and their inhibitory effects on the mRNA expression of inflammatory cytokines and in vivo anti-inflammatory effects on LPS administration were confirmed. A total of 13 compounds were synthesized by coupling tert-butyloxycarbonyl (Boc)-γ-butyric acid to a benzoxazole moiety having various substituents, and then removing the Boc protecting group by hydrolysis under acidic conditions. The biological activities of the 13 final compounds and three intermediates prior to hydrolysis of the Boc protecting group were evaluated.

In order to induce inflammatory responses, lipopolysaccharide (LPS) was used for in vitro and in vivo tests. LPS is a bacterial cell wall component of Gram-negative bacteria and is generally recognized as a potent activator of monocytes/macrophages, the effects of which include altered production of key mediators such as inflammatory cytokines. LPS is also known as a pro-skin inflammation agent, and in many studies, LPS has been used as a stimulus to establish in vitro cellular skin inflammation models.

Bacterial LPS has been widely used in inflammation studies due to the abundance of inflammatory effects generated through TLR signaling. TLRs are transmembrane receptors having an intracellular domain involved in signaling and an extracellular domain interacting with pathogen ligands. In particular, TLRs play an important role in recognizing pathogens in keratinocytes. In the present disclosure, LPS was used to induce inflammation in the human keratinocyte HaCaT cell lines, and the inhibitory effect of novel compounds synthesized according to the Examples above on the expression of inflammatory cytokines was confirmed using the established model.

Accordingly, the present inventors measured the mRNA expression levels of IL-6 and IL-1β in the experimental groups and compared them with the control group. The inhibitory effects of the compounds on IL-6 and IL-1β showed a similar trend. The compounds that were most effective in inhibiting IL-6 mRNA expression (Compounds 5c, 5d, 5f, 5m, and 4d) were also most effective in inhibiting IL-1β mRNA expression. Except for Compound 5m, the compounds with strong activity were compounds in which a methoxy group was not substituted at the 2^{nd} position (R₄) of the benzene ring. Compounds 5f and 4d, which showed high activity on both IL-6 and IL-1β, had a tert-butyl group and a methyl group substituted at the 5^{th} position (R₂) of the benzoxazole, respectively. Compound 4d, an intermediate in which the Boc protecting group was not removed, showed higher anti-inflammatory activity than other intermediates (Compounds 4e and 41). Among the above compounds, when the substituent at the R₂ position was a tert-butyl group, the compounds exhibited excellent activity regardless of the presence of a methoxy group at the R₄ position (Compounds 5f and 5m). When a methyl group was included at the R₂ position, the compounds exhibited excellent activity overall regardless of the presence of a methoxy group at the R₄ position and a Boc protecting group at the R₅ position. When a methyl group was present at the R₃ position, weak activity was observed.

Two compounds were selected from the in vitro test, and an in vivo test was performed to evaluate the inhibitory activity on the expression of IL-6, IL-1β, and TNF-α by injecting LPS into mice. After LPS administration, severe macrophage infiltration was observed in the liver tissue, while administration of Compounds 5f and 4d alleviated the inflammatory response. Furthermore, the present inventors confirmed the mRNA levels of IL-6, IL-1β, and TNF-α in the liver tissue. Compounds 5f and 4d showed significant inhibitory activity on the expression levels of IL-6, IL-1β, and TNF-α. Additionally, the hepatotoxicity of the compounds was confirmed by measuring the ALT and AST levels, and it was confirmed that the groups administered with the compounds showed lower hepatotoxicity compared to the control group administered only with LPS. The present inventors confirmed the strong anti-inflammatory activity of the two compounds through the in vivo study, and no significant difference in activity was observed between the two compounds.

Through LPS-induced inflammatory responses, proinflammatory cytokines IL-6, IL-1β, and TNF-α are activated, and since these cytokines play an important role in inflammatory responses, it is important to effectively control these cytokines.

In the present disclosure, novel benzoxazole derivatives were discovered and it was confirmed that they exhibit anti-inflammatory activity by controlling the expression of inflammatory cytokines. From the results of in vitro and in vivo tests, it was confirmed that the compounds synthesized according to the present disclosure are effective in suppressing representative inflammatory cytokines such as IL-1β, IL-6, and TNF-α. Therefore, through further research, it may be possible to develop small-molecule modulators for inflammatory diseases from these compounds.

Based on the above description, it will be understood by those skilled in the art that the present disclosure may be implemented in a different specific form without changing the technical spirit or essential characteristics thereof. In this regard, it should be understood that the above embodiment is not limitative, but illustrative in all aspects. The scope of the present disclosure is defined by the appended claims rather than by the description preceding them, and therefore all changes and modifications that fall within the metes and bounds of the claims, or equivalents of such metes and bounds are therefore intended to be embraced by the claims.

1. A compound represented by Formula 1 below or a pharmaceutically acceptable salt thereof: 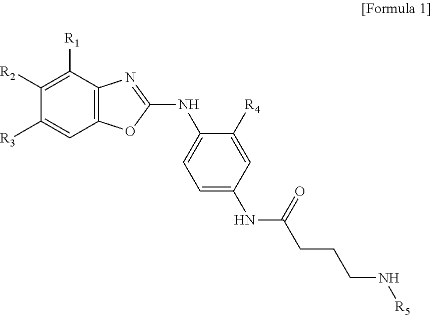
In Formula 1,
R₁ is hydrogen or C₁₋₆ alkyl;
R₂ is hydrogen, halogen, C₁₋₆ alkyl, or C₁₋₆ alkoxy;
R₃ is hydrogen or C₁₋₆ alkyl;
R₄ is hydrogen or C₁₋₆ alkoxy; and
R₅ is hydrogen or C₁₋₆ alkoxycarbonyl.

2. The compound of claim 1 or a pharmaceutically acceptable salt thereof, wherein R₁ is hydrogen or methyl; R₂ is hydrogen, fluoro, chloro, methyl, tert-butyl, or methoxy; R₃ is hydrogen or methyl; R₄ is hydrogen or methoxy; and R₅ is hydrogen or tert-butoxycarbonyl.

3. The compound of claim 1 or a pharmaceutically acceptable salt thereof, wherein the compound is
1. tert-butyl 4-(4-(benzo[d]oxazol-2-ylamino)phenylamino)-4-oxobutylcarbamate;
2. tert-butyl 4-(4-(5-fluorobenzo[d]oxazol-2-ylamino)phenylamino)-4-oxobutylcarbamate;
3. tert-butyl 4-(4-(4-methylbenzo[d]oxazol-2-ylamino)phenylamino)-4-oxobutylcarbamate;
4. tert-butyl 4-(4-(5-methylbenzo[d]oxazol-2-ylamino)phenylamino)-4-oxobutylcarbamate;
5. tert-butyl 4-(4-(6-methylbenzo[d]oxazol-2-ylamino)phenylamino)-4-oxobutylcarbamate;
6. tert-butyl 4-(4-(5-tert-butylbenzo[d]oxazol-2-ylamino)phenylamino)-4-oxobutylcarbamate;
7. tert-butyl 4-(4-(5-methoxybenzo[d]oxazol-2-ylamino)phenylamino)-4-oxobutylcarbamate;
8. tert-butyl 4-(4-(benzo[d]oxazol-2-ylamino)-3-methoxyphenylamino)-4-oxobutylcarbamate;
9. tert-butyl 4-(4-(5-fluorobenzo[d]oxazol-2-ylamino)-3-methoxyphenylamino)-4-oxobutylcarbamate;
10. tert-butyl 4-(4-(5-chlorobenzo[d]oxazol-2-ylamino)-3-methoxyphenylamino)-4-oxobutylcarbamate;
11. tert-butyl 4-(3-methoxy-4-(5-methylbenzo[d]oxazol-2-ylamino)phenylamino)-4-oxobutylcarbamate;
12. tert-butyl 4-(3-methoxy-4-(6-methylbenzo[d]oxazol-2-ylamino)phenylamino)-4-oxobutylcarbamate;
13. tert-butyl 4-(4-(5-tert-butylbenzo[d]oxazol-2-ylamino)-3-methoxyphenylamino)-4-oxobutylcarbamate;
14. 4-amino-N-(4-(benzo[d]oxazol-2-ylamino)phenyl)butanamide;
15. 4-amino-N-(4-(5-fluorobenzo[d]oxazol-2-ylamino)phenyl)butanamide;
16. 4-amino-N-(4-(4-methylbenzo[d]oxazol-2-ylamino)phenyl)butanamide;
17. 4-amino-N-(4-(5-methylbenzo[d]oxazol-2-ylamino)phenyl)butanamide;
18. 4-amino-N-(4-(6-methylbenzo[d]oxazol-2-ylamino)phenyl)butanamide;
19. 4-amino-N-(4-(5-tert-butylbenzo[d]oxazol-2-ylamino)phenyl)butanamide;
20. 4-amino-N-(4-(5-methoxybenzo[d]oxazol-2-ylamino)phenyl)butanamide;
21. 4-amino-N-(4-(benzo[d]oxazol-2-ylamino)-3-methoxyphenyl)butanamide;
22. 4-amino-N-(4-(5-fluorobenzo[d]oxazol-2-ylamino)-3-methoxyphenyl)butanamide;
23. 4-amino-N-(4-(5-chlorobenzo[d]oxazol-2-ylamino)-3-methoxyphenyl)butanamide;
24. 4-amino-N-(3-methoxy-4-(5-methylbenzo[d]oxazol-2-ylamino)phenyl)butanamide;
25. 4-amino-N-(3-methoxy-4-(6-methylbenzo[d]oxazol-2-ylamino)phenyl)butanamide; or
26. 4-amino-N-(4-(5-tert-butylbenzo[d]oxazol-2-ylamino)-3-methoxyphenyl)butanamide.

4. A method for preparing the compound of claim 1 or a pharmaceutically acceptable salt thereof, comprising:
a first step of reacting a compound represented by Formula 2 below with tert-butyl 4-amino-4-oxobutylcarbamate to prepare a compound represented by Formula 3 below; and
a second step of optionally hydrolyzing in the presence of an acid to remove the tert-butoxycarbonyl protecting group: 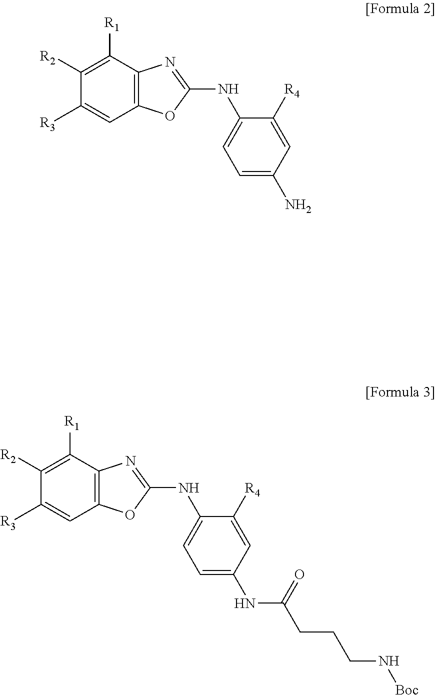
In the Formula 2 or 3,
R₁ to R₄ are as defined in claim 1.

5. The method of claim 4, wherein the first step is performed in the presence of a peptide coupling reagent and Hunig's base.

6. A pharmaceutical composition for preventing or treating an inflammatory disease, comprising a compound represented by Formula 1 or a pharmaceutically acceptable salt thereof, as an active ingredient: 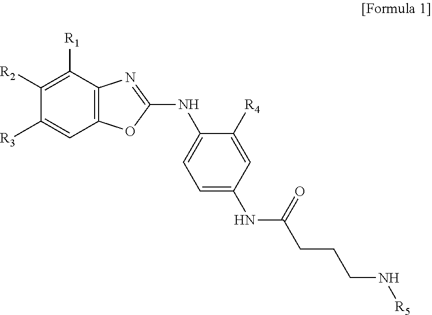
In Formula 1,
R₁ is hydrogen or C₁₋₆ alkyl;
R₂ is hydrogen, halogen, C₁₋₆ alkyl, or C₁₋₆ alkoxy;
R₃ is hydrogen;
R₄ is hydrogen or C₁₋₆ alkoxy; and
R₅ is hydrogen or C₁₋₆ alkoxycarbonyl.

7. The composition of claim 6, wherein R₁ is hydrogen or methyl; R₂ is hydrogen, fluoro, chloro, methyl, tert-butyl, or methoxy; R₃ is hydrogen; R₄ is hydrogen or methoxy; and R₅ is hydrogen or tert-butoxycarbonyl.

8. The composition of claim 6, wherein the compound is
1. tert-butyl 4-(4-(benzo[d]oxazol-2-ylamino)phenylamino)-4-oxobutylcarbamate;
2. tert-butyl 4-(4-(5-fluorobenzo[d]oxazol-2-ylamino)phenylamino)-4-oxobutylcarbamate;
3. tert-butyl 4-(4-(4-methylbenzo[d]oxazol-2-ylamino)phenylamino)-4-oxobutylcarbamate;
4. tert-butyl 4-(4-(5-methylbenzo[d]oxazol-2-ylamino)phenylamino)-4-oxobutylcarbamate;
5. tert-butyl 4-(4-(5-tert-butylbenzo[d]oxazol-2-ylamino)phenylamino)-4-oxobutylcarbamate;
6. tert-butyl 4-(4-(5-methoxybenzo[d]oxazol-2-ylamino)phenylamino)-4-oxobutylcarbamate;
7. tert-butyl 4-(4-(benzo[d]oxazol-2-ylamino)-3-methoxyphenylamino)-4-oxobutylcarbamate;
8. tert-butyl 4-(4-(5-fluorobenzo[d]oxazol-2-ylamino)-3-methoxyphenylamino)-4-oxobutylcarbamate;
9. tert-butyl 4-(4-(5-chlorobenzo[d]oxazol-2-ylamino)-3-methoxyphenylamino)-4-oxobutylcarbamate;
10. tert-butyl 4-(3-methoxy-4-(5-methylbenzo[d]oxazol-2-ylamino)phenylamino)-4-oxobutylcarbamate;
11. tert-butyl 4-(4-(5-tert-butylbenzo[d]oxazol-2-ylamino)-3-methoxyphenylamino)-4-oxobutylcarbamate;
12. 4-amino-N-(4-(benzo[d]oxazol-2-ylamino)phenyl)butanamide;
13. 4-amino-N-(4-(5-fluorobenzo[d]oxazol-2-ylamino)phenyl)butanamide;
14. 4-amino-N-(4-(4-methylbenzo[d]oxazol-2-ylamino)phenyl)butanamide;
15. 4-amino-N-(4-(5-methylbenzo[d]oxazol-2-ylamino)phenyl)butanamide;
16. 4-amino-N-(4-(5-tert-butylbenzo[d]oxazol-2-ylamino)phenyl)butanamide;
17. 4-amino-N-(4-(5-methoxybenzo[d]oxazol-2-ylamino)phenyl)butanamide;
18. 4-amino-N-(4-(benzo[d]oxazol-2-ylamino)-3-methoxyphenyl)butanamide;
19. 4-amino-N-(4-(5-fluorobenzo[d]oxazol-2-ylamino)-3-methoxyphenyl)butanamide;
20. 4-amino-N-(4-(5-chlorobenzo[d]oxazol-2-ylamino)-3-methoxyphenyl)butanamide;
21. 4-amino-N-(3-methoxy-4-(5-methylbenzo[d]oxazol-2-ylamino)phenyl)butanamide; or
22. 4-amino-N-(4-(5-tert-butylbenzo[d]oxazol-2-ylamino)-3-methoxyphenyl)butanamide.

9. The composition of claim 6, which modulates the TLR signaling pathway by reducing mRNA expression of inflammatory cytokines in vitro or in vivo.

10. The composition of claim 9, wherein the inflammatory cytokine is one or more selected from the group consisting of IL-6, IL-1β, and TNF-α.
